(19) United States
(12) Patent Application Publication
(10) Pub. No.: US 2024/0384325 A1
(43) Pub. Date: Nov. 21, 2024

(54) SPLIT AND POOL IDENTIFICATION OF RBP TARGETS (SPIDR)

(71) Applicants: California Institute of Technology, Pasadena, CA (US); The Trustees of Columbia University in the City of New York, New York, NY (US)

(72) Inventors: Erica Wolin, New York, NY (US); James Guo, Pasadena, CA (US); Mitchell Guttman, Los Angeles, CA (US); Marko Jovanovic, New York, NY (US)

(21) Appl. No.: 18/665,401

(22) Filed: May 15, 2024

Related U.S. Application Data
(60) Provisional application No. 63/466,761, filed on May 16, 2023.

(51) Int. Cl. C12Q 1/6804; C12Q 1/6806; C12Q 1/6834; C12Q 1/6869

(52) U.S. Cl. CPC C12Q 1/6804; C12Q 1/6806; C12Q 1/6834; C12Q 1/6869

(57) ABSTRACT

Aspects of the present disclosure generally relate to methods and compositions for detecting an association between a RNA binding protein and a RNA. Some aspects of the present disclosure relate to methods of generating an antibody-bead conjugate pool. Some aspects of the present disclosure relate to kits and compositions for performing the methods disclosed herein.

### INCORPORATION BY REFERENCE TO ANY PRIORITY APPLICATIONS

This application claims the benefit of U.S. Provisional App. No. 63/466,761, filed May 16, 2023, which is incorporated by reference in its entirety herein. Any and all applications for which a foreign or domestic priority claim is identified in the Application Data Sheet as filed with the present application are hereby incorporated by reference under 37 CFR 1.57.

### STATEMENT REGARDING FEDERALLY SPONSORED R&D

This invention was made with government support under Grant No(s). HG012216 & AG071869 & GM128802 awarded by the National Institutes of Health and Grant No. MCB2224211 awarded by the National Science Foundation. The government has certain rights in the invention.

### FIELD

Embodiments herein generally relate to methods, compositions, and kits, for detecting associations between RNA binding proteins (RBPs) and other molecules, for example RBP-RNA interactions.

### BACKGROUND

RNA binding proteins (RBPs) play key roles in controlling all stages of the mRNA life cycle, including transcription, processing, nuclear export, translation, and degradation. Recent estimates suggest that up to 30% of all human proteins (several thousand in total) bind to RNA, indicative of their broad activity and central importance in cell biology. Moreover, mutations in RBPs have been causally linked to various human diseases, including immunoregulatory and neurological disorders as well as cancer. Despite their importance, the specific roles of most RBPs remain unexplored because it is unknown what specific RNAs most RBPs bind.

In addition, there are many thousands of regulatory non-coding RNAs (ncRNAs) whose functional roles remain largely unknown; understanding how they work requires defining the proteins to which they bind. For example, uncovering the mechanism by which the Xist long noncoding RNA (lncRNA) silences the inactive X chromosome required identification of the SPEN/SHARP RBP that binds to Xist—a process that took >25 years after the lncRNA was discovered. Given the large discrepancy between the number of ncRNAs and putative RBPs identified, and the number of RNA-protein interactions demonstrated to be functionally relevant, there is an urgent need to generate high-resolution binding maps to enable functional characterization.

Currently, the most rigorous and widely utilized method to characterize RBP-RNA interactions is crosslinking and immunoprecipitation followed by next generation sequencing (CLIP-seq). Briefly, CLIP works by utilizing UV light to covalently crosslink RNA and directly interacting proteins, followed by cell lysis, immunoprecipitation under stringent conditions (e.g., 1M salt) to purify a protein of interest followed by gel electrophoresis, transfer to a nitrocellulose membrane, and excision of the protein-RNA complex prior to sequencing and identification of the bound RNAs. CLIP and its related variants have greatly expanded our knowledge of RNA-RBP interactions and our understanding of gene expression from mRNA splicing to microRNA targeting.

Yet, CLIP and nearly all of its variants are limited to mapping a single RBP at a time. As such, efforts to generate reference maps for hundreds of RBPs in even a limited number of cell types have required major financial investment and the work of large teams working in international consortiums (e.g., ENCODE). Despite these efforts and the important advances they have enabled, there are critical limitations: (i) Only a small fraction of the total number of predicted RBPs have been successfully mapped using genome-wide methods; (ii) Of these, most have been mapped in only a small number of cell lines (mainly K562 and HepG2); (iii) Because each protein map is generated from an individual experiment, a large number of cells is required to map dozens, let alone hundreds, of RBPs—this is particularly challenging for studying primary cells, disease models, or other populations of rare cells. Further, because these datasets are highly cell type-specific, the generated maps are not likely to be directly useful for studying these RBPs within other cell-types or model systems (e.g., patient samples, animal models, or perturbations). Thus, it is important to enable the generation of comprehensive RBP binding information for any cell type of interest. Accordingly, some aspects of the present disclosure are directed to methods and compositions for detecting associations between RBPs and RNA.

### SUMMARY

Some aspects of the present disclosure relate to methods of detecting an association between an RNA binding protein (RBP) and an RNA. In some embodiments, the methods comprise providing an antibody-bead conjugate pool. In some embodiments, the antibody-bead conjugate pool comprises two or more different antibody-bead conjugate populations. In some embodiments, each antibody-bead conjugate population comprises a plurality of antibody-bead conjugates. In some embodiments, each antibody-bead conjugate in an antibody-bead conjugate population comprises an antibody specific for a single RNA binding protein and a first oligonucleotide that identifies the RNA binding protein recognized by the antibody. In some embodiments, each antibody in an antibody-bead conjugate population is specific for the same RBP. In some embodiments, each antibody in an antibody-bead conjugate population is the same antibody. In some embodiments, each antibody-bead conjugate population in the antibody-bead conjugate pool comprises a different antibody as compared to one or more other antibody-bead conjugate populations in the antibody-population conjugate pool. In some embodiments, each antibody-bead conjugate population in the antibody-bead conjugate pool comprises an antibody specific for a different RBP. In some embodiments, the antibody and RBP-identifying oligonucleotide are separately conjugated to the bead. In some embodiments, the bead pool comprises a biotinylated protein G bead bound to a streptavidin-biotin-tag complex.

In some embodiments the methods further comprise providing a sample comprising a plurality of RNA binding proteins and a plurality of RNA molecules. In some embodiments, the RNA and RNA binding proteins are cross-linked to form RNA: RBP complexes. In some embodiments, a plurality of RNA molecules are each crosslinked to a single RBP. In some embodiments, at least one RBP is crosslinked to a plurality of RNA. The sample may be obtained from one or more cells or tissues.

In some embodiments the methods further comprise immunopurifying cross-linked RNA: RBP complexes using the antibody-bead conjugate pool. Following immunopurification, split-and-pool barcoding of the immunopurified molecules is performed. In some embodiments, multiple rounds of split and pool barcoding are performed, for example, but not limited to, 5 rounds or more of split and pool barcoding. During each of the one or more rounds of split-and-pool barcoding the same barcode oligonucleotide is added to both the RBP-identifying oligonucleotide and the immunopurified RNA in an RNA: RBP complex. In some embodiments, the barcodes added in consecutive rounds of split-and-pool barcoding are different, such that specific barcodes are created for the RNA and RBP-identifying oligonucleotide on each antibody-bead conjugate. The barcoded molecules are then sequenced. The RNA from the immunopurified RNA: RBP complexes are then associated with their corresponding RBP by matching the RBP-identifying oligonucleotide and RNA based on their shared barcode.

Some aspects of the present disclosure relate to methods of generating antibody-bead pools. In some embodiments, the methods comprise conjugating the same RBP-identifying oligonucleotide to a plurality of beads to generate a first bead pool. In some embodiments, the beads are protein A, protein G, or protein A/G beads. In some embodiments, the bead is a magnetic bead. In some embodiments, the bead is biotinylated. Beads that are generally suitable for conjugation to an antibody or binding fragment are known in the art. A plurality of different bead pools may be generated, each labeled with a different antibody-identifying oligonucleotide. An antibody to an RNA binding protein, or a binding fragment thereof, is then conjugated to each bead in the first bead pool to generate a first antibody-bead conjugate population. A second antibody is conjugated to each bead in a second bead pool to generate a second antibody-bead conjugate population. Additional antibody-bead conjugate populations may be generated. In some embodiments, the first and second antibodies are specific for different RBPs. In some embodiments, the first and second antibodies are different antibodies. A plurality of different antibody-bead conjugate populations may be generated. In some embodiments, each antibody in a given antibody-conjugate population is the same antibody. In some embodiments, each antibody in a given antibody-conjugate population is specific to the same RBP. The plurality of antibody-bead conjugate populations are then pooled to generate an antibody-bead conjugate pool. In some embodiments, the methods comprise incubating one or more populations of biotinylated protein G beads with a streptavidin-biotin oligo complex, wherein each population of beads is labeled with a different oligonucleotide; incubating each of the one or more populations of beads with an antibody, wherein each population of beads is incubated with a different antibody; and combining each of the one or more populations of beads to generate an antibody-bead conjugate pool.

Some embodiments disclosed herein relate to antibody-bead conjugate populations. In some embodiments, the antibody-bead conjugate populations comprise a plurality of antibody-bead conjugates. In some embodiments, each bead in the antibody-bead conjugate population is conjugated to an antibody or binding fragment thereof specific for a RNA binding protein and with an RBP-identifying oligonucleotide. Each antibody or binding fragment thereof in the antibody-bead conjugate population is specific for the same RNA binding protein. Each bead in an antibody-bead conjugate population is labeled with the same RBP-identifying oligonucleotide. In some embodiments, the bead comprises a protein A, protein G, or protein A/G bead. In some embodiments, the bead is a magnetic bead.

Some embodiments disclosed herein relate to antibody-bead conjugate pools. In some embodiments, the antibody-bead conjugate pools comprise a plurality of antibody-bead conjugate populations. Each antibody-bead conjugate population is conjugated to an antibody or binding fragment thereof specific for a single RNA binding protein and an antibody-identifying oligonucleotide. Each antibody or binding fragment thereof in an antibody-bead conjugate population is specific for the same RNA binding protein. Each bead in an antibody-bead conjugate population is labeled with the same RBP-identifying oligonucleotide. Each different antibody-bead conjugate population is specific for a different RBP. In some embodiments, the bead comprises a protein A, protein G, or protein A/G bead. In some embodiments, the bead is a magnetic bead. In some embodiments, the antibody-bead conjugate pool comprises biotinylated protein G beads bound to a streptavidin-biotin-tag complex.

Some aspects of the present disclosure relate to kits for detecting interactions between RBPs and RNA. In some embodiments, the kits comprise a labeled bead pool. In some embodiments, the beads are protein A, protein G, or protein A/G beads. In some embodiments, the bead is a magnetic bead. In some embodiments, the bead is biotinylated. In some embodiments, the labeled bead pool comprises plurality of oligonucleotide labeled bead populations. In some embodiments, each bead in a labeled bead population comprises an antibody or binding fragment specific to an RBP and an RBP-identifying oligonucleotide, where the RBP-identifying oligonucleotide corresponds to the antibody. In some embodiments, each labeled bead population in a labeled bead pool comprises a different RBP-identifying oligonucleotide, corresponding to the RBP antibody in that bead population.

In some embodiments, the kits comprise an antibody-bead conjugate pool comprising a plurality of antibody-bead conjugate populations. In some embodiments, each antibody-bead conjugate population comprises a plurality of antibody-bead conjugates, where each antibody-bead conjugate comprises an antibody to an RBP and an oligonucleotide linked to the bead. In some embodiments, each antibody in an antibody-bead conjugate population is specific for the same RNA binding protein. In some embodiments, each antibody in an antibody-bead conjugate population is the same antibody. In some embodiments, each antibody-bead conjugate population in an antibody-bead conjugate pool is specific for a different RBP. In some embodiments each oligonucleotide in an antibody-bead conjugate population is the same. In some embodiments, the antibody-bead conjugate pool comprises a biotinylated protein G bead bound to a streptavidin-biotin-tag complex. In some embodiments, the kit further comprises one or more barcode oligonucleotides, for example, up to 100 unique barcode oligonucleotides. In some embodiments, the kit further comprises a cross-linking agent.

### BRIEF DESCRIPTION OF THE DRAWINGS

In addition to the features described above, additional features and variations will be readily apparent from the following descriptions of the drawings and exemplary embodiments. It is to be understood that these drawings depict various embodiments and aspects and are not intended to be limiting in scope.

FIG. 1A is a schematic overview of some embodiments of the Split and Pool Identification of RBP targets (SPIDR) method.

FIG. 1B is a schematic list of some embodiments of different RBPs mapped by SPIDR in K562 and/or HEK293T cells, functional assignments based on literature review.

FIG. 1C is an example of some embodiments of a raw alignment data for a pool (all reads before splitting by bead identities) and for specific RBPs (all reads assigned to specific RBP beads) across the XIST RNA. Blocks represent exons, lines introns, and thick blocks are the annotated XIST repeat regions (A-E).

FIG. 1D depicts some embodiments of a raw alignment data for some SLBP across the H3C2 histone mRNA. Top track depicts some embodiments of pooled alignment data; tracks below depict some embodiments of reads assigned to SLBP or other RBPs and controls.

FIG. 2A depicts some embodiments of RNA binding patterns of selected RBPs (rows) relative to 100 nt windows across each classical non-coding RNA (columns). Each bin is colored based on the enrichment of read coverage per RBP relative to background.

FIG. 2B depicts some embodiments of sequence read coverage for LSM11 binding to U7 snRNA. For all tracks, “pool” refers to all reads prior to splitting them by paired barcodes (shown in light gray), and individual tracks (shown in dark gray) reflect reads after assignment to specific antibodies.

FIG. 2C depicts some embodiments of enrichment of read coverage relative to background for WDR43 and LIN28B over the 5′ ETS region of 45S RNA.

FIG. 2D depicts some embodiments of sequence reads coverage for LIN28B binding to let-7 miRNAs.

FIG. 2E depicts some embodiments of sequence reads coverage for DROSHA/DGCR8, UPF1, SPEN, and TARDBP to their respective mRNAs.

FIG. 2F depicts some embodiments of sequence reads coverage for two distinct antibodies to HNRNPL in a single SPIDR experiment. For comparison, HNRNPL coverage from the ENCODE-generated eCLIP data is also shown.

FIG. 3A-G depicts some embodiments of examples of concordant binding identified by eCLIP (ENCODE consortium) and SPIDR. Sequence reads coverage is shown for individual proteins measured by ENCODE and SPIDR along with a negative control (IgG). FIG. 3A depicts some embodiments of concordant binding of HNRNPK. FIG. 3B discloses some embodiments of concordant binding of PTBP1. FIG. 3C depicts some embodiments of concordant binding of RBFOX2. FIG. 3D depicts some embodiments of a comparison of ENCODE and SPIDR data for multiple proteins bound to the XIST lncRNA. Sequence reads coverage for PTBP1, HNRNPU (SAF-A), and HNRNPK are shown. FIG. 3E depicts some embodiments of the significance of overlap between binding sites detected by SPIDR and those identified within paired proteins in the ENCODE data. Each bin represents the paired protein between both experiments, blue represent a hypergeometric p-value of less than 0.01. FIG. 3F depicts some embodiments of peak annotation in matched SPIDR and ENCODE data. Stacked bar plot showing the percentage of peaks detected in the SPIDR(S) or ENCODE (E) datasets in various annotation categories. FIG. 3G depicts some embodiments of a comparison of significant motifs identified within SPIDR peaks (right, p-value threshold <1e-40) to those reported for RNA Bind-n-Seq (left) or eCLIP (middle).

FIG. 4A is a schematic showing how reverse transcription pause sites can be used to map RBP-RNA interactions at single nucleotide resolution. UV light crosslinks the RBP to the target RNA at points of direct contact. During reverse transcription, the enzyme preferentially stalls at the crosslinking site, leading to termination of cDNA synthesis (STOP). Mapping the 3′-end of the cDNA (truncations) may identify the RBP binding site at single nucleotide resolution.

FIG. 4B depicts some embodiments of the SPIDR determined binding sites of RPS2 and RPS6 overlayed on the known 80S ribosome structure. SPIDR data shown is from HEK293T cells.

FIG. 4C depicts some embodiments of HNRNPC binding sites for STRN3 (left) and MRPL52 (right). Both raw read alignments (“Reads”, top) and 3′-end truncations of the cDNA (“Truncations”, bottom) are shown. The upper two panels show the mapped reads and truncations for the whole gene, the lower two panels are zoomed-in on the indicated region.

FIG. 4D depicts some examples of PTBP1 binding sites for PTBP1 itself (left) and XIST (right). Both raw read alignments and 3′-end truncations of the cDNA reads are shown.

FIG. 4E depicts some embodiments of truncation frequency (3′ ends of the mapped cDNA reads) over all significantly enriched HNRNPC peaks is shown centered on the motif position. The region of the steep frequency rise of truncations is shown by the line on the y-axis and corresponds to the displayed sequence.

FIG. 4F depicts some embodiments of the truncation frequency (3′ ends of the mapped cDNA reads) over all significantly enriched PTBP1 peaks. Truncation frequency is shown relative to the motif position within each peak. The region of the steep frequency rise of truncations is shown by the line on the y-axis and corresponds to the displayed sequence.

FIG. 5A depicts some embodiments of the frequency of 3′-end truncations of LARP1 reads plotted across the 18S rRNA. Zoom-in shows accumulation near nucleotide position 1700 (indicated by the gray bar). Data shown is from HEK293T cells.

FIG. 5B depicts some embodiments of the structure of the 40S ribosomal subunit bound to the 5′-end of an mRNA molecule. The first nucleotide of the mRNA is indicated as labeled. RPS2 and RPS3 are indicated for orientation and the mRNA is indicated as labeled. green. The LARP1 binding site detected on the 18S rRNA is indicated as labeled.

FIG. 5C depicts some embodiments of examples of LARP1 binding for three different mRNAs containing TOP motifs in their 5′UTRs-TPT1 (left), RPS8 (middle) and EEF1G (right). Both read alignments (“Reads”, top) and 3′end truncations of the cDNA reads (“Truncations”, bottom) are shown. TOP motifs within the 5′-UTRs are depicted as indicated by gray bars on the X-axis.

FIG. 5D depicts some embodiments of a model of LARP1 interactions based on SPIDR data. LARP1 shows preferential binding to both the 18S rRNA (close to the mRNA entry channel of the 40S subunit) and the TOP motifs within the 5′UTR of specific mRNAs. In this way, LARP1 could facilitate recruitment of the 40S subunit to TOP motif-containing mRNAs.

FIG. 6A depicts some embodiments of a schematic of an experimental approach for the mTOR perturbation experiment. HEK293T cells are treated with either 250 nM torin or control (solvent only) for 18 hours. SPIDR is performed on both samples. The multiplexed IP is performed separately, and the samples are mixed after the first round of barcoding.

FIG. 6B depicts some embodiments of cumulative Distribution Function (CDF) plots of protein changes in torin versus control treated samples as determined by LC-MS/MS. log 2 ratios (Torin/Control) are shown on the x-axis and fraction of total (from 0 to 1) is shown on the y-axis. Proteins were grouped into four categories based on their TOP motif score as previously published in (Philippe et al., 2020). The analysis was performed on the 2000 most highly expressed genes (based on RNA expression).

FIG. 6C depicts some embodiments of the number of SPIDR reads assigned to each RBP in the torin-treated samples versus control samples. 4EBP1, EIF4A, LARP1 and LARP4 are also indicated. Dashed line corresponds to enrichment of 1.

FIG. 6D depicts some embodiments of raw alignment data for selected RBPs across RPS2, an mRNA with a strong TOP motif. For each protein “control” and “torin” treatment tracks are shown.

FIG. 6E depicts some embodiments of violin plots of the log 2 ratios (torin/control) of significant binding sites for 4EBP1. The RNA targets are grouped based on their TOP motif score as published in (Philippe et al., 2020). Asterisks indicate statistical significance (p-value <0.00001, Mann-Whitney).

FIG. 6F depicts some embodiments of violin plots of the log 2 ratios (Torin/Control) of significant binding sites for LARP1. The RNA targets were grouped based on their TOP motif score as published in (Philippe et al., 2020) 60. Asterisks indicate statistical significance (p-value <0.00001, Mann-Whitney).

FIG. 6G depicts some embodiments of a model of mTOR-dependent repression of mRNA translation. LARP1 binds to the 40S ribosome and to 5′ untranslated region of TOP-containing mRNAs independent of mTOR activity. When mTOR is active (i.e., in the absence of torin; right side), this dual binding modality can recruit the ribosome specifically to TOP-containing mRNAs and promote their translation. When mTOR is inactive (i.e., in the presence of torin), 4EBP1 can bind to TOP-containing mRNAs (possibly through an interaction with LARP1) and to EIF4E. The interaction between 4EBP1 and EIF4E prevents binding between EIF4E and EIF4G, which is required to initiate translation. In this way, LARP1/4EBP1 binding specifically to TOP-containing mRNAs would enable sequence-specific repression of translation.

FIG. 7 depicts some embodiments of a schematic for a multiplexed antibody-bead labeling strategy. Populations of biotinylated protein G beads are incubated with a streptavidin-biotin oligo complex. Each population of beads is labeled with an oligo with a specific sequence and then incubated with one type of capture antibody such that each population has a unique capture antibody and a corresponding oligo tag that can be recognized after sequencing. Populations are combined to create the bead pool.

FIG. 8 depicts some embodiments of a scatter plot showing log 2 transformed IBAQ (intensity based absolute quantification) values for all identified proteins in either the pooled IP with 39 targets (y-axis) versus those detected with a V5 negative control IP (x-axis) by LC-MS/MS. Target proteins that should be detected by the antibodies included in the pool of 39 used are marked in dark gray.

FIG. 9 depicts some embodiments of observed distributions of labeled beads after sequencing. Each bead is defined in sequencing by a particular, unique combinatorial barcode acquired during split-pool. A SPIDR cluster represents any set of molecules, oligo or RNA, that share the same bead combinatorial barcode. Left: CDF plot showing some embodiments of the number of independent oligos matched within an individual SPIDR cluster. Right: CDF plot describing some embodiments of the degree of heterogeneity of these detected oligos within each SPIDR cluster, as determined by oligos with a shared combinatorial barcode. X axis represents the homogeneity of the oligo types with 1 indicating that all oligos are of the same type.

FIG. 10 depicts some embodiments of the number of deduplicated mapped reads and number of significant binding sites within uniquely mapped genomic regions per IP. The order is determined by the number of unique mapped reads in both plots.

FIG. 11 depicts some embodiments of an example of a background correction method that utilizes the total read coverage across all proteins to normalize each individual protein. Shown are example tracks on RN7SK before and after background correction. Left: Raw alignment data for the entire pooled dataset (top track) and for representative antibodies against U2AF1, TARDBP, SHARP, LARP7 and HNRNPK on RN7SK. Right: Background corrected data for the same set of antibodies. Signal that was not antibody-specific is normalized out. The reads in the right are binned in 5 nucleotide windows. RN7SK is known to be bound by LARP7.

FIG. 12 depicts some embodiments of an auto-regulatory binding matrix with protein (x-axis) binding to each mRNA (y-axis). Each target protein included in SPIDR performed in K562 cells marked by whether it has significantly enriched binding within its own RNA, or in any of the other SPIDR target RNAs. Proteins that bind their own RNA are marked in black, instances of binding to genes of other SPIDR targets are marked in gray.

FIG. 13A depicts some embodiments of heatmaps showing the percentage of significant binding sites in each of the annotation categories for SPIDR performed in K562 cells and ENCODE.

FIG. 13B depicts some embodiments of a quantitative assessment of the similarity of heatmaps between SPIDR and ENCODE. The Euclidean distance (L2 norm) between the ENCODE and SPIDR percentage tables/heatmaps is calculated. The calculated distance is indicated by the dashed line. The statistical significance is calculated by randomly shuffling the columns of either the SPIDR percentage table and keeping the original ENCODE table or vice versa, meaning shuffling the columns of the ENCODE table and keeping the original SPIDR table. This is done 1000 times in each direction and every time the Euclidean distance was calculated. The values are represented by the two histograms. The Euclidean distance of all of the randomly shuffled 2000 comparison is always larger than of the true pair, which shows that the two original annotation tables from SPIDR and ENCODE are highly significantly similar (p-value <0.0005).

FIG. 14 is a table depicting some embodiments of an overview of the SPIDR experiment in K562 cells. The protein targets are listed, as well as the vendors and product numbers of the corresponding antibodies.

FIG. 15 is a table depicting some embodiments of an overview of the SPIDR experiment HEK293T cells treated with Torin or Control (solvent only). The protein targets are listed, as well as the vendors and product numbers of the corresponding antibodies.

### DETAILED DESCRIPTION

Disclosed herein are methods and compositions to identify RBP-RNA interactions. The methods may be referred to as SPIDR (Split and Pool Identification of RBP targets). In some embodiments the methods provide a massively multiplexed way to generate high-quality, high-resolution, transcriptome-wide maps of RBP-RNA interactions. SPIDR can map RBPs with a wide-range of RNA binding characteristics and functions (including, e.g., mRNAs, lncRNAs, rRNAs, small RNAs, etc.) and enables the study of diverse RNA processes (e.g., splicing, translation, miRNA processing, etc.) within a single experiment and at an unprecedented scale.

In some embodiments, SPIDR is able to simultaneously profile the global RNA binding sites of dozens to hundreds of RBPs in a single experiment, thus enabling rapid, de novo discovery of RNA-protein interactions at an unprecedented scale.

SPIDR is based on a split-pool barcoding strategy that maps multiway nucleic acid interactions using high throughput sequencing. In some embodiments a vastly simplified version of split-pool barcoding presented herein, when combined with antibody-bead barcoding, increases throughput relative to current CLIP methods by two orders of magnitude. In some embodiments the methods allow for reliable identification of the precise, single nucleotide RNA binding sites of RBPs, and in some embodiments the precise binding sites of dozens of RBPs can be identified simultaneously. In some embodiments the methods allow for the detection of changes in RBP binding upon perturbation.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The term “polynucleotide,” refers to a polymeric form of nucleotides of any length, including DNA, RNA, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

A “nucleic acid” sequence refers to a deoxyribonucleic acid (DNA) or ribonucleic acid (RNA) sequence. The term captures sequences that include any of the known base analogues of DNA and RNA such as, but not limited to 4-acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, the term “antibody” denotes the meaning ascribed to it by one of skill in the art, and further it is intended to include any polypeptide chain-containing molecular structure with a specific shape that fits to and recognizes an epitope, where one or more non-covalent binding interactions stabilize the complex between the molecular structure and the epitope. Antibodies utilized in the present invention may be polyclonal antibodies, although monoclonal antibodies are preferred because they may be reproduced by cell culture or recombinantly and can be modified to reduce their antigenicity.

In addition to entire immunoglobulins (or their recombinant counterparts), immunoglobulin fragments or “binding fragments” comprising the epitope binding site (e.g., Fab′, F(ab′)2, single-chain variable fragment (scFv), diabody, minibody, nanobody, single-domain antibody (sdAb), or other fragments) are useful as antibody moieties in the present invention. Such antibody fragments may be generated from whole immunoglobulins by ricin, pepsin, papain, or other protease cleavage. Minimal immunoglobulins may be designed utilizing recombinant immunoglobulin techniques. For instance, “Fv” immunoglobulins for use in the present invention may be produced by linking a variable light chain region to a variable heavy chain region via a peptide linker (e.g., poly-glycine or another sequence which does not form an alpha helix or beta sheet motif). Nanobodies or single-domain antibodies can also be derived from alternative organisms, such as dromedaries, camels, llamas, alpacas, or sharks. In some embodiments, antibodies can be conjugates, e.g., pegylated antibodies, drug, radioisotope, or toxin conjugates. Monoclonal antibodies directed against a specific epitope, or combination of epitopes, will allow for the targeting and/or depletion of cellular populations expressing the marker. Various techniques can be utilized using monoclonal antibodies to screen for cellular populations expressing the marker(s) and include magnetic separation using antibody-coated magnetic beads, “panning” with antibody attached to a solid matrix (i.e., plate), and flow cytometry (e.g., U.S. Pat. No. 5,985,660, hereby expressly incorporated by reference in its entirety).

As known in the art, the term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally comprises two constant domains, CH2 and CH3. As is known in the art, an Fc region can be present in dimer or monomeric form.

As known in the art, a “constant region” of an antibody refers to the constant region of the antibody light chain or the constant region of the antibody heavy chain, either alone or in combination.

A “variable region” of an antibody refers to the variable region of the antibody light chain or the variable region of the antibody heavy chain, cither alone or in combination. As known in the art, the variable regions of the heavy and light chains each consist of four framework regions (FRs) connected by three complementarity determining regions (CDRs) also known as hypervariable regions and contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonical class as the subject variable region (Chothia and Lesk, J Mol Biol 196 (4): 901-917, 1987).

As used herein, the term “antigen binding molecule” refers to a molecule that comprises an antigen binding portion that binds to an antigen and, optionally, a scaffold or framework portion that allows the antigen binding portion to adopt a conformation that promotes binding of the antigen binding portion or provides some additional properties to the antigen binding molecule. In some embodiments, the antigen is Gal3. In some embodiments, the antigen binding portion comprises at least one CDR from an antibody that binds to the antigen. In some embodiments, the antigen binding portion comprises all three CDRs from a heavy chain of an antibody that binds to the antigen or from a light chain of an antibody that binds to the antigen. In some embodiments, the antigen binding portion comprises all six CDRs from an antibody that binds to the antigen (three from the heavy chain and three from the light chain). In some embodiments, the antigen binding portion is an antibody fragment.

Non-limiting examples of antigen binding molecules include antibodies, antibody fragments (e.g., an antigen binding fragment of an antibody), antibody derivatives, and antibody analogs. Further specific examples include, but are not limited to, a single-chain variable fragment (scFv), a nanobody (e.g. VH domain of camelid heavy chain antibodies; VHH fragment, see Cortez-Retamozo et al., Cancer Research, Vol. 64:2853-57, 2004), a Fab fragment, a Fab′ fragment, a F(ab′)2 fragment, a Fv fragment, a Fd fragment, and a complementarity determining region (CDR) fragment. These molecules can be derived from any mammalian source, such as human, mouse, rat, rabbit, pig, dog, cat, horse, donkey, guinea pig, goat, or camelid. Antibody fragments may compete for binding of a target antigen with an intact antibody and the fragments may be produced by the modification of intact antibodies (e.g., enzymatic, or chemical cleavage) or synthesized de novo using recombinant DNA technologies or peptide synthesis. The antigen binding molecule can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding molecule as well as wholly synthetic scaffolds comprising, for example, a biocompatible polymer. Sec, for example, Korndorfer et al., 2003, Proteins: Structure, Function, and Bioinformatics, Volume 53, Issue 1:121-129 (2003); Roque et al., Biotechnol. Prog. 20:639-654 (2004). In addition, peptide antibody mimetics (“PAMs”) can be used, as well as scaffolds based on antibody mimetics utilizing fibronectin components as a scaffold.

An antigen binding molecule can also include a protein comprising one or more antibody fragments incorporated into a single polypeptide chain or into multiple polypeptide chains. For instance, antigen binding molecule can include, but are not limited to, a diabody (see, e.g., EP 404,097; WO 93/11161; and Hollinger et al., Proc. Natl. Acad. Sci. USA, Vol. 90:6444-6448, 1993); an intrabody; a domain antibody (single VL or VH domain or two or more VH domains joined by a peptide linker; see Ward et al., Nature, Vol. 341:544-546, 1989); a maxibody (2 scFvs fused to Fc region, see Fredericks et al., Protein Engineering, Design & Selection, Vol. 17:95-106, 2004 and Powers et al., Journal of Immunological Methods, Vol. 251:123-135, 2001); a triabody; a tetrabody; a minibody (scFv fused to CH3 domain; see Olafsen et al., Protein Eng Des Sel., Vol. 17:315-23, 2004); a peptibody (one or more peptides attached to an Fc region, see WO 00/24782); a linear antibody (a pair of tandem Fd segments (VH—CH1-VH-CH1) which, together with complementary light chain polypeptides, form a pair of antigen binding regions, see Zapata et al., Protein Eng., Vol. 8:1057-1062, 1995); a small modular immunopharmaceutical (see U.S. Patent Publication No. 20030133939); and immunoglobulin fusion proteins (e.g. IgG-scFv, IgG-Fab, 2scFv-IgG, 4scFv-IgG, VH-IgG, IgG-VH, and Fab-scFv-Fc).

In certain embodiments, an antigen binding molecule can have, for example, the structure of an immunoglobulin. An “immunoglobulin” is a tetrameric molecule, with each tetramer comprising two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

As used herein, a “composition” refers to any mixture of two or more products, substances, or compounds, including cells. It may be a formulation, solution, a suspension, liquid, powder, a paste, aqueous, non-aqueous or any combination thereof.

As used herein, the term “kit” may be used to describe variations of a portable, self-contained enclosure that includes at least one set of components to conduct one or more of the methods of the invention.

As used herein “crosslinking forces” and “crosslinking agents” have their customary and ordinary meaning as would be understood by one of ordinary skill in the art in view of this disclosure. These terms refer to forces and agents that can induce the formation of covalent bonds between substances that are in proximity to each other, for example, a query protein associated with target moiety as described herein. Advantageously, the crosslinking forces and agents of some embodiments may be used in vivo, so that a query protein and associated target moiety in vivo may be covalently bound together, and remain covalently bound together after they are recovered from the in vivo environment. It is contemplated that by crosslinking query proteins and target moieties in vivo, bona fide associations between the query proteins and target moieties can be detected. Subsequent non-covalently interacting-substances (such as artifacts of contact with other substances or sample materials or contaminants) can be removed under denaturing conditions as described herein. In contrast, and without being limited by theory, in vitro methods to identify intermolecular interactions (for example performed in cell extracts) may identify artifactual associations, for example between molecules that are expressed in different cell types or different cellular compartments, or at different times, and are unlikely to actually associate in vivo.

In methods, compositions, and kits of some embodiments, the crosslinking agent or force comprises ultraviolet radiation, or an amine-to-amine crosslinker (such as disuccinimidyl suberate or disuccinimidyl tartrate), or a sulfhydryl-to-sulfhydryl crosslinker (such as bis-maleimidoethane or dithio-bis-maleimidoethane), or an aryl-azide (such as N-5-Azido-2-nitrobenzyloxysuccinimide or sulfosuccinimidyl 6-(4′-azido-2′-nitrophenylamino) hexanoate), or a diazirine (such as succinimidyl 4,4′-azipentanoate). By way of example, a crosslinking agent may comprise an agent selected from the group consisting of an NHS ester, an imidoester, a difluoro group, an NHS-haloacetyl group, an NHS-maleimide group, an NHS-pyridyldithiol group, a carbodiimide ester and NHS ester, a malemide and a hydrazine group, a pyridyldithiol and a hydrazine group, a NHS ester and an aryl azide, a NHS ester and a diazirine, a NHS ester and an aryl azide, and a diazirine, or a combination of two or more of any of the listed items. In methods, compositions, and kits of some embodiments, the crosslinking agent or force comprises ultraviolet radiation, or an amine-to-amine crosslinker (such as disuccinimidyl suberate or disuccinimidyl tartrate), or a sulfhydryl-to-sulfhydryl crosslinker (such as bis-maleimidoethane or dithio-bis-maleimidoethane), or an aryl-azide (such as N-5-Azido-2-nitrobenzyloxysuccinimide or sulfosuccinimidyl 6-(4′-azido-2′-nitrophenylamino) hexanoate), or a diazirine (such as succinimidyl 4,4′-azipentanoate), or an NHS ester, an imidoester, a difluoro group, an NHS-haloacetyl group, an NHS-maleimide group, an NHS-pyridyldithiol group, a carbodiimide ester and NHS ester, a malemide and a hydrazine group, a pyridyldithiol and a hydrazine group, a NHS ester and an aryl azide, a NHS ester and a diazirine, a NHS ester and an aryl azide, and a diazirine, or a combination of two or more of any of the listed items.

As used herein, “barcode” has its customary and ordinary meaning as would be understood by one of ordinary skill in the art in view of this disclosure. It may refer to an identifier that can be associated with a RBP-identifying oligonucleotide and an immunopurified RNA in an RNA: RBP complex. For example, a barcode can comprise an oligonucleotide sequence, and/or a detectable moiety or combinations of oligonucleotide sequences and/or detectable moieties (such as fluorophores, nanoparticles, and/or quantum dots). In some embodiments a barcode is a combinatorial barcode. In some embodiments, a barcode comprises at least 5 nucleotides, for example, at least 5, 6, 7, 8, 9, 10, 11, 12, 13, 14, 15, 16, 17, 18, 19, 20, 21, 22, 23, 24, 25, 25, 27, 28, 29, or 30 nucleotides, including ranges between any two of the listed values, for example 5-10, 5-15, 5-20, 5-25, 5-30, 7-10, 7-15, 7-20, 7-25, 7-30, 10-15, 10-20, 10-25, 10-30, 12-15, 12-20, 12-25, 12-30, 15-20, 15-25, 15-30, 20-25, or 20-30 nucleotides. In some embodiments a barcode comprises a plurality of barcode individual barcodes that have been added individually during split and pool processing. Barcodes may also contain additional nucleic acid sequences, for example universal primer annealing sites, which can facilitate sequencing.

As used herein, “combinatorial barcode” has its customary and ordinary meaning as would be understood by one of ordinary skill in the art in view of this disclosure. It may refer to a type of barcode that comprises multiple “combinatorial barcode units” or “barcode oligonucleotides,” which together yield the combinatorial barcode. For example, each combinatorial barcode unit or barcode oligonucleotide can comprise an oligonucleotide subunit, and the sequence of the oligonucleotide subunit can provide identification information for the combinatorial barcode unit. In some embodiments, each combinatorial barcode unit can comprise an oligonucleotide subunit and a detectable moiety or combination of detectable moieties (such as a fluorophore, nanoparticle, quantum dot, or the like), which provide identifying information for the combinatorial barcode. For example, the combinatorial barcode can comprise a polyfluorophore. By way of example, a combinatorial barcode unit or barcode oligonucleotide may comprise, consist essentially of, or consist of an oligonucleotide of at least 3, 4, 5, 6, 7, 8, 9, 10, 11, 12, 13, 14, 15,16, 17, 18, 19, or 20 nucleotides in length, including ranges between any two of the listed values, for example, 3-8, 3-12, 3-16, or 3-20, 4-8, 4-12, 4-16, 4-20, 6-8, 6-12, 6-16, 6-20, 10-12, 10-16, or 10-20 nucleotides. The number of different combinatorial barcode units, and the length of the combinatorial barcode may depend on the scale of the detecting method or kit. A combinatorial barcode may comprise at least 2 combinatorial barcode units, for example, at least 2, 3, 4, 5, 6, 7, 8, 9, 10, 11, 12, 13, 14, 15, 16, 17, 18, 19, or 20, including ranges between any two of the listed values, for example, 2-8, 2-12, 2-16, 2-20, 3-8, 3-12, 3-16, 3-20, 4-8, 4-12, 4-16, 4-20, 6-8, 6-12, 6-16, 6-20, 10-12, 10-16, or 10-20 combinatorial barcode units.

As used herein, “split-and-pool barcoding” has its customary and ordinary meaning as would be understood by one of ordinary skill in the art in view of this disclosure. It may refer to barcoding in which a composition comprising molecules is split into two or more partitions that are separate from each other. Then, the composition of each partition is barcoded so that molecules in the same partition are barcoded with the same barcode unit, but molecules in different partitions are barcoded with different barcode unitss from each other. After the barcoding, the contents of the partitions can be pooled to form a composition. The process can be repeated on this composition, so that multiple iterations of splitting, barcoding, and pooling are performed. The term “partitions” refer to spaces that are in fluid isolation from each other, so that the contents of the different partitions do not mix while they are in the partitions. For example, the partitions can be separated by one or more solid barriers. Examples of partitions include, but are not limited to, wells of a multi-well plate (e.g., 96-well plate), containers such as microcentrifuge tubes, chambers of a fluid device, and the like.

After multiple iterations of split-and-pool barcoding, the macromolecules, for example RNA, and candidate interaction partners, for example, RNA binding proteins, will each comprise a combination of combinatorial barcode units. These combinations may be referred to as “combinatorial barcodes” or simply as “barcodes” (and accordingly, the barcoding to produce the combinatorial barcodes may be referred to as “combinatorial barcoding.”).

As used herein, the singular forms “a,” “an,” and “the” include plural referents unless the context clearly dictates otherwise. For example, “a” or “an” means “at least one” or “one or more.” It is understood that aspects, embodiments, and variations described herein include “comprising,” “consisting,” and/or “consisting essentially of aspects, embodiments and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

### Non-Limiting Embodiments

Some embodiments disclosed herein relate to methods of detecting an association between a RNA binding protein (RBP) and a RNA. In some embodiments, the methods enable highly multiplexed mapping of RBPs to individual RNAs transcriptome wide. Briefly, in some embodiments, the methods involve: (i) generating multiplexed antibody-bead pools by tagging individual antibody-bead conjugates with a specific oligonucleotide (tagged bead pools), (ii) performing RBP purification using these tagged antibody-bead pools in crosslinked cell lysates, and (iii) linking individual RBP to their associated RNAs using split- and -pool barcoding.

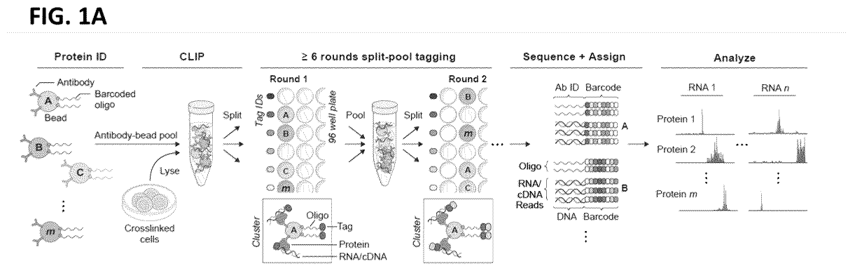

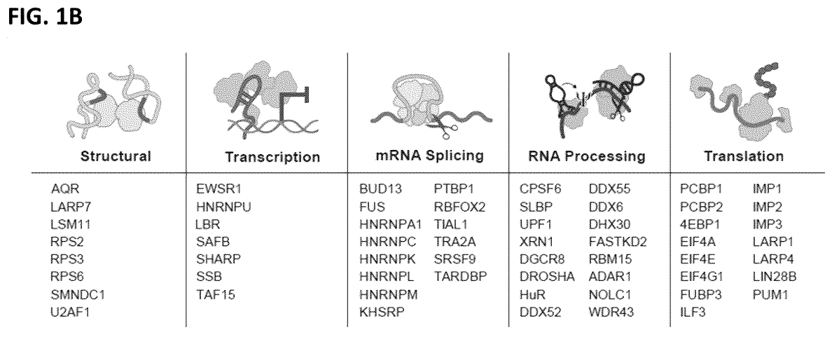

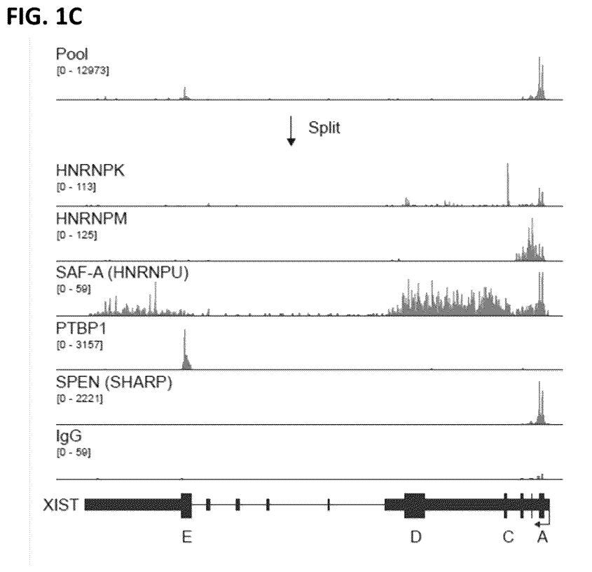

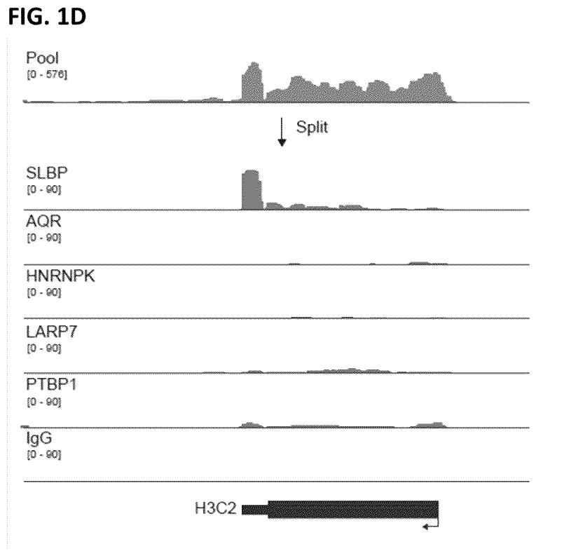

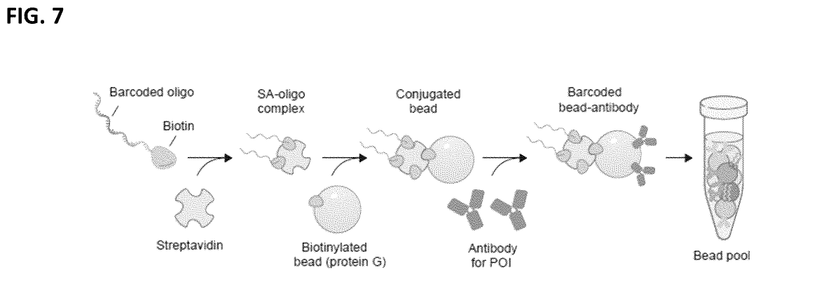

As discussed herein, a highly modular scheme is provided that allows for the generation of hundreds of tagged antibody-bead conjugates. The tagged antibody-bead conjugates form a pool comprising multiple different antibody-bead conjugate populations, where each unique antibody-bead population comprises beads labeled with a specific oligonucleotide tag and antibodies to a specific RBP. Multiple antibody-bead populations can be combined to generate an antibody-bead pool (FIG. 1A and FIG. 7).

In some embodiments, the methods comprise providing an antibody-bead conjugate pool. In some embodiments, the antibody-bead conjugate pool comprises two or more different antibody-bead conjugate populations. In some embodiments, each antibody-bead conjugate population comprises a plurality of antibody-bead conjugates. In some embodiments, each antibody-bead conjugate in an antibody-bead conjugate population comprises an antibody, antigen binding molecule, or an antigen binding fragment thereof, specific for a single RNA binding protein and an oligonucleotide that identifies the RNA binding protein recognized by the antibody, antigen binding molecule, or an antigen binding fragment thereof. In some embodiments, the oligonucleotide is a RBP-identifying oligonucleotide, where the sequence of the oligonucleotide is associated with the antibody, antigen binding molecule, or an antigen binding fragment thereof, on the bead. As the antibody, antigen binding molecule, or an antigen binding fragment thereof, is in turn specific for a particular RBP, the oligonucleotide also identifies the RBP bound by the antibody, antigen binding molecule, or an antigen binding fragment thereof. In some embodiments, each bead in a population of beads is labeled with the same oligonucleotide. In some embodiments, each bead population is labeled with a different oligonucleotide. In some embodiments, each bead population is labeled with one or more oligonucleotides that are associated with antibody, antigen binding molecule, or an antigen binding fragment thereof, specific to a different RBP, such that the oligonucleotides of each bead population are associated with a specific RBP. In some embodiments, each antibody, antigen binding molecule, or an antigen binding fragment thereof, in an antibody-bead conjugate population is specific for the same RBP. In some embodiments, each antibody, antigen binding molecule, or an antigen binding fragment thereof, in an antibody-bead conjugate population is the same antibody, antigen binding molecule, or an antigen binding fragment thereof. In some embodiments, each antibody-bead conjugate population in the antibody-bead conjugate pool comprises a different antibody, antigen binding molecule, or an antigen binding fragment thereof, as compared to one or more other antibody-bead conjugate populations in the antibody-population conjugate pool. In some embodiments, each antibody-bead conjugate population in the antibody-bead conjugate pool comprises an antibody, antigen binding molecule, or an antigen binding fragment thereof, specific for a different RBP. In some embodiments, the antibody, antigen binding molecule, or an antigen binding fragment thereof, and RBP-identifying oligonucleotide are separately conjugated to the bead. In some embodiments, the bead pool comprises a biotinylated protein G bead bound to a streptavidin-biotin-tag complex. Antibodies, antigen binding molecules, and binding fragments thereof, are generally known in the art. Because the methods disclosed herein do not require direct chemical modification of the antibody, antigen binding molecule, or an antigen binding fragment thereof, any known and/or commercially available antibody, antigen binding molecule, or an antigen binding fragment thereof, (in any storage buffer) may be used and rapidly associated with a defined oligonucleotide sequence on a bead at high efficiency.

Non-limiting examples of antigen binding molecules suitable for use in the methods disclosed herein include antibodies, antibody fragments (e.g., an antigen binding fragment of an antibody), antibody derivatives, and antibody analogs. Further specific examples include, but are not limited to, a single-chain variable fragment (scFv), a nanobody (e.g. VH domain of camelid heavy chain antibodies; VHH fragment, see Cortez-Retamozo et al., Cancer Research, Vol. 64:2853-57, 2004), a Fab fragment, a Fab′ fragment, a F(ab′)2 fragment, a Fv fragment, a Fd fragment, and a complementarity determining region (CDR) fragment. In some embodiments the antigen binding molecule is derived from a mammalian source, such as human, mouse, rat, rabbit, pig, dog, cat, horse, donkey, guinea pig, goat, or camelid. As used herein, the term “antibody” and “antigen binding molecule” may be used interchangeably.

In some embodiments, the oligonucleotide is conjugated to the bead before the antibody. In some embodiments, the oligonucleotide is conjugated to the bead after the antibody. In some embodiments, the antibody is conjugated to the bead using the same coupling procedure utilized in traditional CLIP-based approaches. In some embodiments, antibodies are conjugated to protein A, protein G, or protein A/G beads. In some embodiments, the antibody is covalently conjugated to the bead. In some embodiments, the antibody is non-covalently conjugated to the bead. In some embodiments, the bead is a magnetic bead. In some embodiments, the bead is biotinylated. Beads generally suitable for conjugation to an antibody are known in the art. Many such beads are commercially available, for example, but not limited to, Dynabeads. It is expected that one skilled in the art would recognize that any known and/or commercially available bead may be used in the methods disclosed herein. Two or more populations are combined to create the bead pool.

There are a number of suitable methods for attaching oligonucleotides or barcodes to beads, RNA, and/or other oligonucleotides or macromolecules as described herein. For example, the beads, RNA, and/or other oligonucleotides or macromolecules can be barcoded using one or more techniques, such as genetic conjugation of a nucleic acid to a polypeptide (e.g., boxB-lambdaN system), mRNA display methods, or direct conjugation of nucleic acids to polypeptides). In some embodiments, for example, if a bead comprises an identifier barcode as described herein, combinatorial barcode units can be directly added to the oligonucleotide. Methods for coupling of oligonucleotides to proteins are also described, for example, in in Los et al., “HaloTag: a novel protein-labeling technology for cell imaging and protein analysis, ACS Chem Biol., 2008, 3:373-382; Blackstock et al., “Halo-Tag Mediated Self-Labeling of Fluorescent Proteins to Molecular Beacons for Nucleic Acid Detection,” Chem. Commun., 2014, 50:1375-13738; Kozlov et al., “Efficient Strategies for the Conjugation of Oligonucleotides to Antibodies Enabling Highly Sensitive Protein Detection,” Biopolymers, 2004, 73:621; and Solulink, “Antibody-Oligonucleotide Conjugate Preparation,” Solulink.com, 4 pages, each of which is incorporated by reference in its entirety herein.

Using the antibody-bead conjugate pool, RBPs crosslinked to one or more RNAs are purified from a sample. Purification may be carried out by, for example, but not limited to, on-bead immunoprecipitation (IP), of RBPs crosslinked to one or more RNAs.

In some embodiments, the RNA is messenger RNA (mRNA), ribosomal RNA (rRNA), signal recognition particle RNA (7SL RNA or SRP RNA), transfer RNA (tRNA), transfer-messenger RNA (tmRNA), small nuclear RNA (snRNA), small nucleolar RNA (snoRNA), SmY RNA (SmY), small cajal body-specific RNA (scaRNA), guide RNA (gRNA), ribonuclease P (RNase P), ribonuclease MRP (RNase MRP), Y RNA, telomerase RNA component (TERC), spliced leader RNA (SL RNA), antisense RNA (aRNA, asRNA), cis-natural antisense transcript (cis-NAT), CRISPR RNA (crRNA), non-coding RNA (ncRNA), long non-coding RNA (lncRNA), microRNA (miRNA), piwi-interacting RNA (piRNA), small interfering RNA (siRNA), short hairpin RNA (shRNA), trans-acting siRNA (tasiRNA), repeat associated siRNA (rasiRNA), 7SK RNA (7SK), enhancer RNA (eRNA), or any combination thereof. In some embodiments, the RNA is naturally occurring. In some embodiments, the RNA is synthetic.

In some embodiments, RNA in the sample is crosslinked to RBPs in the sample to form one or more RBP: RNA complexes. In some embodiments, one or more crosslinking agents or forces are used to cross link the RNA to the RBP. Methods of crosslinking a nucleic acid to a protein are known in the art. Such methods are suitable for use in the methods of the present disclosure. In some embodiments, the crosslinking agent or force comprises ultraviolet radiation, or an amine-to-amine crosslinker (such as disuccinimidyl suberate or disuccinimidyl tartrate), or a sulfhydryl-to-sulfhydryl crosslinker (such as bis-maleimidoethane or dithio-bis-maleimidoethane), or an aryl-azide (such as N-5-Azido-2-nitrobenzyloxysuccinimide or sulfosuccinimidyl 6-(4′-azido-2′-nitrophenylamino) hexanoate), or a diazirine (such as succinimidyl 4,4′-azipentanoate), or an NHS ester, an imidoester, a difluoro group, an NHS-haloacetyl group, an NHS-maleimide group, an NHS-pyridyldithiol group, a carbodiimide ester and NHS ester, a malemide and a hydrazine group, a pyridyldithiol and a hydrazine group, a NHS ester and an aryl azide, a NHS ester and a diazirine, a NHS ester and an aryl azide, and a diazirine, or any combination thereof.

In some embodiments, purification of crosslinked RBP: RNA complexes comprises providing a sample comprising, or suspected of comprising, one or more RNA binding proteins and a plurality of RNA molecules. In some embodiments, the sample is a biological sample. In some embodiments the biological sample comprises a plurality of cells. In some embodiments, the biological sample is from a healthy source. In some embodiments, the biological sample is from a diseased source. In some embodiments, the biological sample may comprise a cell culture, a cell line, a cell extract, a cell lysate, whole tissue, a tissue extract, a tissue sample, such as, for example, a biopsy, a whole organ, a tumor, a tumor cell, a cell mass, a tumor cell or tumor cell extract, a pre-cancerous lesion, polyp, or cyst, a cellular component or compartment, neuronal dendrites, suspension cells, adherent cells, transformed cells, tissue culture cells, primary cell lines, or any combination thereof. In some embodiments, the biological sample is disrupted, disaggregated, homogenized, or lysed by any technique known in the art. For example, the biological sample may be made into a single-cell suspension using a nylon filter or mesh. Cells or tissue comprising the biological sample may, in one embodiment, be adhered to a substrate such as a chip, a slide, a dish, etc. In some embodiments, the cells are washed according to techniques known to one skilled in the art.

Individual RNA binding protein identities are assigned to their associated RNAs using split-and-pool barcoding. In each split-pool round, pools of crosslinked RNA: RBP complexes bound to corresponding beads are randomly split and distributed into two or more partitions that are separate from each other. Then, each bead and RNA in each partition is barcoded so that the RBP-specific oligonucleotide and RNA associated with each antibody-bead conjugate in each well are labeled with the same well-specific barcode (FIG. 1A), but molecules in different partitions are barcoded with different barcodes from each other. After the barcoding, the contents of the partitions can be pooled to form a composition. The process can be repeated on this composition, so that multiple iterations of splitting, barcoding, and pooling are performed. The process is repeated in multiple barcoding rounds, with a unique barcode added in each round. The complexity of the final combinatorial barcode generated during the split-and-pool barcoding process depends on the number of individual barcode tags or units used in each split-pool round and the number of split-pool rounds. For example, after 8 rounds of split and pool barcoding, using 12 barcodes in each round, the likelihood that two beads will end up with the same combinatorial barcode is ˜1 in 430 million (1/128).

There are a number of suitable methods for barcoding beads and/or RNA with a combinatorial barcode unit in accordance with the methods and kits disclosed herein. For example, in some embodiments, each combinatorial barcode unit can comprise a common “handle” oligonucleotide sequence (which may also be referred to as a “linker”) and the complement of the handle, which may link combinatorial barcode units to a growing combinatorial barcode and/or each other. The handle and complement of the handle can be disposed on opposite termini of the combinatorial barcode unit. The growing combinatorial barcode can thus comprise a single-stranded complement of the handle, and each added combinatorial barcode unit can hybridize, through its handle, to the growing combinatorial barcode, while leaving a complement of the handle available for adding additional combinatorial barcode units. The hybridized combinatorial barcode unit and growing combinatorial barcode can then be ligated. In the methods and kits of some embodiments disclosed herein, the handles and complements of the handles are single-stranded. In the methods and kits of some embodiments, the handles are comprised 3′ ends of primers that anneal to growing ends of combinatorial barcode subunit. Upon extension, the primer can produce an oligonucleotide that comprises the sequences of the combinatorial barcode thus far, along with a handle for the additional of an additional combinatorial barcode subunit. In the methods of some embodiments, the handle comprises at least 4, 5, 6, 7, 8, 9, 10, 11, 12, 13, 14, or 15 nucleotides, including ranges between any two of the listed values, such as 4-8, 4-10, 4-15, 8-10, 8-15, or 10-15 nucleotides. Examples of combinatorial barcoding methods are described, for example, in U.S. Pre-Grant Publication No. 2019/0187156, which is incorporated by reference in its entirety herein.

After multiple iterations of split-and-pool barcoding, the beads and RNA will each comprise a combination of barcode units. These combinations may be referred to as “combinatorial barcodes” (and accordingly, the barcoding to produce the combinatorial barcodes may be referred to as “combinatorial barcoding.”).

Following the split-and-pool barcoding, oligonucleotides and RNA molecules and their linked barcodes are sequenced and RNAs are matched to RBPs based on shared combinatorial barcodes and the known relationship between the oligonucleotide and the RBP antibodies. That is, if an RNA and oligonucleotide share the same combinatorial barcode, a relationship between the RNA and the RBP associated with the oligonucleotide is determined.

In some embodiments, the number of barcoding rounds performed for each SPIDR experiment is determined based on the complexity of the given bead pool. In some embodiments, the split-and-pool barcode ligation steps are performed for 1, 2, 3, 4, 5, 6, 7, 8, 9, or 10 minutes at room temperature. In some embodiments, one or more agents to prevent RNA degradation are added to the samples during the split-and-pool ligation steps. Compared to previously published approaches, the number of barcodes required per round is reduced. The number of rounds of split-and-pool barcoding may increase as the ligation step is optimized. Therefore, the barcoding procedure is significantly simplified in contrast to previous versions.

In some embodiments, multiple rounds of split-and-pool barcoding are performed. In some embodiments, 1, 2, 3, 4, 5, 6, 7, 8, 9, 10, 15, 20, 25, 30, 40, 50 or more rounds of split-and-pool barcoding are performed, or a number of rounds of split-and-pool barcoding that is in a range defined by any two of the preceding values. For example, in some embodiments, between 1-50, 1-40, 1-30, 1-25, 1-15, 1-10, 1-8, 1-6, 1-4, 1-2, 2-50, 2-40, 2-30, 2-25, 2-20, 2-10, 2-8, 2-6, 2-4, 4-50, 4-40, 4-30, 4-25, 4-20, 4-10, 4-8, 4-6, 6-50, 6-40, 6-340, 6-25, 6-20, 6-10, 6-8, 8-50, 8-40, 8-30, 8-25, 8-20, 8-10, 10-50, 10-40, 10-30, 10-25, 10-20, 20-50, 20-40, 20-30, or 25-50, 25-30, 30-50, 30-40, or 40-50 rounds of split-and-pool barcoding are performed. In some embodiments, at least 6 rounds of split-and-pool barcoding are performed. In some embodiments, at least 8 rounds of split-and-pool barcoding are performed. In some embodiments, more than 10 rounds of split-and-pool barcoding are performed. For example, in some embodiments, up to 25 rounds of split-and-pool barcoding are performed. In some embodiments, more than 25 rounds of split-and-pool barcoding are performed.

In some embodiments, multiple unique barcode oligonucleotides are used in each round of split-and-pool barcoding. For example, in some embodiments, 1, 2, 3, 4, 5, 6, 7, 8, 9, 10, 20, 25, 30, 40, 50, 60, 70, 75, 80, 90, or 100 unique barcode oligonucleotides are used in each round of split-and-pool barcoding, or the number of unique barcode oligonucleotides used is in a range defined by any two of the preceding values. For example, in some embodiments, between 1-100, 1-75, 1-50, 1-25, 1-10, 1-5, 5-100, 5-75, 5-50, 5-25, 5-10, 10-100, 10-75, 10-50, 10-25, 25-100, 25-75, 25-50, 50-100, 50-75, or 75-100, unique barcode oligonucleotides are used.

In some embodiments, multiple unique barcodes are used over multiple rounds of split-and-pool barcoding. In some embodiments, at least 1, 2, 3, 4, 5, 6, 7, 8, 9, or 10 rounds, or a number of rounds in a range defined by any two of the preceding values, of split-and-pool barcoding using at least 1, 2, 3, 4, 5, 6, 7, 8, 9, 10, 20, 25, 30, 40, 50, 60, 70, 75, 80, 90, or 100 unique barcode oligonucleotides per round, or a number of unique barcode oligonucleotides in a range that is defined by any two of the preceding values, are performed. For example, in some embodiments, between 1-10, 1-8, 1-6, 1-4, 1-2, 2-10, 2-8, 2-6, 2-4, 4-10, 4-8, 4-6, 6-10, 6-8, or 8-10 rounds of split-and-pool barcoding using between 1-100, 1-75, 1-50, 1-25, 1-10, 1-5, 5-100, 5-75, 5-50, 5-25, 5-10, 10-100, 10-75, 10-50, 10-25, 25-100, 25-75, 25-50, 50-100, 50-75, or 75-100, unique barcode oligonucleotides per round are performed. In some embodiments, at least 6 rounds of split-and-pool barcoding using at least 24 barcodes per round are performed. In some embodiments, at least 6 rounds of split-and-pool barcoding using at least 36 barcodes per round are performed. In some embodiments, at least 8 rounds of split-and-pool barcoding using at least 24 barcodes per round are performed. In some embodiments, at least 8 rounds of split-and-pool barcoding using at least 36 barcodes per round are performed.

In some embodiments, following split-and-pool barcoding, the barcoded molecules are converted to complementary DNA (cDNA). In some embodiments, the cDNA is then fragmented, end-repaired, and made into sequencing libraries. Sequencing libraries are pools of DNA fragments containing adapter sequences compatible with a specific sequencing platform and indexing barcodes for individual sample identification. Library preparation methods are known in the art. Any such method is suitable for use in the methods disclosed herein. Exemplary library preparation methods include, but are not limited to, ligation-based library preparation, tagmentation-based library preparation, and amplicon library preparation. The specific library preparation protocol used depends on many factors including the sequencing platform and desired downstream analysis. The basic steps of library preparation are fragmentation and end repair, addition of adapters, and (optional), PCR amplification.

Following split-and-pool barcoding and library preparation, the barcoded antibody-bead conjugates and RNA are sequenced. Methods for sequencing nucleic acids are known in the art. Any such method may be suitable for use in the methods disclosed herein. Following sequencing, all antibody-bead tags and RNA reads are matched by their shared barcodes; these are referred to herein as “SPIDR clusters” (FIG. 1A). SPIDR clusters may be merged by protein identity (specified by the antibody-bead oligonucleotide) to generate a high-depth binding map for each protein. The resulting datasets are analogous to those generated by traditional individual CLIP approaches.

In some embodiments, each SPIDR cluster comprises 1, 2, 3, 4, 5, 6, 7, 8, 9, or 10 barcodes, or a number of barcodes that is in a range defined by any two of the preceding values. For example, in some embodiments, each SPIDR cluster comprises between 1-10, 1-7, 1-5, 1-3, 1-2, 2-10, 2-7, 2-5, 2-3, 3-10, 3-7, 3-5, 5-10, 5-7, or 7-10 barcodes. In some embodiments, about 70%, 75%, 80%, 85%, 90%, 95%, or 100% of barcodes, or a percentage of barcodes in a range defined by any two of the preceding values, in a SPIDR cluster identify a single RBP. For example, in some embodiments, between about 70%-100%, 70%-95%, 70%-90%, 70%-80%, 70%-75%, 75%-100%, 75%-95%, 75%-90%, 75%-85%, 80%-100%, 80%-95%, 80%-90%, 90%-100%, 90%-95%, or 95%-100% of barcodes in a SPIDR cluster identify a single RBP. In some embodiments, the specificity of one or more SPIDR clusters enables assignment of RNA molecules to their corresponding RBPs. In some embodiments, PCR duplicates, i.e., sequences sharing identical start and stop genetic positions are removed as part of the assignment process. In some embodiments, high confidence binding sites are identified by comparing read coverage across a RNA to the read coverage of all other targets in composition.

In some embodiments, the methods disclosed herein generate single nucleotide contact maps that accurately recapitulate the RNA-protein contacts observed within structural models. In some embodiments, the methods disclosed herein generate single nucleotide contact maps that recapitulate the RNA-protein contacts observed within structural models with at least about 70%, 75%, 80%, 85%, 90%, 95%, or 100% accuracy, or with an accuracy that is in a range defined by any two of the preceding values. For example, in some embodiments, the methods disclosed herein generate single nucleotide contact maps that recapitulate the RNA-protein contacts observed within structural models with at least between about 70%-100%, 70%-95%, 70%-90%, 70%-80%, 70%-75%, 75%-100%, 75%-95%, 75%-90%, 75%-85%, 80%-100%, 80%-95%, 80%-90%, 90%-100%, 90%-95%, or 95%-100% accuracy.

In some embodiments, the methods disclosed herein comprise providing an antibody-bead conjugate pool. In some embodiments, the antibody-bead conjugate pool comprises a plurality of antibody-bead conjugate populations. Each antibody-bead conjugate population comprises a plurality of antibody-bead conjugates. In some embodiments, each antibody-bead conjugate comprises an antibody specific for a single RBP and an antibody identifying oligonucleotide. Each antibody-bead conjugate population in the antibody-conjugate pool is specific for a different RBP. In some embodiments, the method further comprises providing a composition comprising RNA crosslinked to a plurality RBPs. In some embodiments, the composition comprises a plurality of non-crosslinked RNA and RBP. In some embodiments, the methods further comprise crosslinking the RNA and RBP to form and RNA: RBP complex. In some embodiments, one or more crosslinking agents or forces are used. The crosslinked RNA: RBP complex is then immunopurified using the antibody-bead conjugate pool. Following purification, one or more rounds of split-and-pool barcoding are performed. During each of the one or more rounds of split-and-pool barcoding, the same barcode oligonucleotide is added to both antibody identifying oligonucleotide and the RNA. The barcoded molecules are then sequenced. RNA are assigned to their associated RBP based on their shared barcodes. In some embodiments, the bead pool comprises a biotinylated protein G bead bound to a streptavidin-biotin-tag complex. In some embodiments, the composition comprises a cell. In some embodiments, the methods disclosed herein further comprise lysing the cell. In some embodiments, the composition comprises a cell lysate. In some embodiments, assigning the RNA to their corresponding RBP comprises matching the antibody-bead conjugate and RNA based on their shared barcode oligonucleotides. In some embodiments, immunopurifying the cross-linked RNA using the antibody-bead conjugate pool enriches the two or more RNA binding proteins relative to a negative control. In some embodiments, the generates single nucleotide contact maps that accurately recapitulate the RNA-protein contacts observed within structural models.

Some embodiments disclosed herein relate to methods of generating an antibody-bead conjugate pool. In some embodiments, the methods comprise: incubating one or more populations of biotinylated protein G beads with a streptavidin-biotin oligo complex, wherein each population of beads is labeled with a barcode oligonucleotide; incubating each of the one or more populations of barcoded beads with an antibody, wherein each population of beads is incubated with a different antibody; and combining each of the one or more populations of beads to generate an antibody-bead conjugate pool.

In some embodiments, a composition comprising two or more different RNA binding proteins and one or more RNA is disclosed. In some embodiments, the RNA is messenger RNA (mRNA), ribosomal RNA (rRNA), signal recognition particle RNA (7SL RNA or SRP RNA), transfer RNA (tRNA), transfer-messenger RNA (tmRNA), small nuclear RNA (snRNA), small nucleolar RNA (snoRNA), SmY RNA (SmY), small cajal body-specific RNA (scaRNA), guide RNA (gRNA), ribonuclease P (RNase P), ribonuclease MRP (RNase MRP), Y RNA, telomerase RNA component (TERC), spliced leader RNA (SL RNA), antisense RNA (aRNA, asRNA), cis-natural antisense transcript (cis-NAT), CRISPR RNA (crRNA), non-coding RNA (ncRNA), long non-coding RNA (lncRNA), microRNA (miRNA), piwi-interacting RNA (piRNA), small interfering RNA (siRNA), short hairpin RNA (shRNA), trans-acting siRNA (tasiRNA), repeat associated siRNA (rasiRNA), 7SK RNA (7SK), enhancer RNA (cRNA), or any combination thereof. In some embodiments, the RNA is naturally occurring. In some embodiments, the RNA is synthetic.

In some embodiments, a composition comprising two or more different RNA binding proteins and one or more RNA is disclosed. In some embodiments, the composition comprises 2, 3, 4, 5, 6, 7, 8, 9, 10, 20, 25, 30, 40, 50, 60, 70, 75, 80, 90,100,200, 250, 300, 400, 500, 600, 700, 750, 800, 900, 1000 or more RNA binding proteins, or the composition comprises a number of RNA binding proteins that is in a range defined by any two of the preceding values. For example, in some embodiments, the composition comprises between 1-1000, 1-750, 1-500, 1-250, 1-100, 1-75, 1-50, 1-25, 1-10, 1-5, 5-1000, 5-750, 5-500, 5-250, 5-100, 5-75, 5-50, 5-25, 5-10, 10-1000, 10-750, 10-500, 10-250, 10-100, 10-75, 10-50, 10-25, 25-1000, 25-750, 25-500, 25-250, 25-100, 25-75, 25-50, 50-1000, 50-750, 50-500, 50-250, 50-100, 100-1000, 100-750, 100-500, 100-250, 250-1000, 250-750, 250-500, 500-1000, 500-750, or 750-1000, RNA binding proteins. In some embodiments, the composition comprises more than 1000 RNA binding proteins.

In some embodiments, a composition comprising two or more different RNA binding proteins and one or more RNA is disclosed. In some embodiments, the composition comprises 1, 2, 3, 4, 5, 6, 7, 8, 9, 10, 20, 25, 30, 40, 50, 60, 70, 75, 80, 90, 100, 200, 250, 300, 400, 500, 600, 700, 750, 800, 900, 1000 or more, different RNA, or the composition comprises a number of different RNA that is in a range defined by any two of the preceding values. For example, in some embodiments, the composition comprises between 1-1000, 1-750, 1-500, 1-250, 1-100, 1-75, 1-50, 1-25, 1-10, 1-5, 5-1000, 5-750, 5-500, 5-250, 5-100, 5-75, 5-50, 5-25, 5-10, 10-1000, 10-750, 10-500, 10-250, 10-100, 10-75, 10-50, 10-25, 25-1000, 25-750, 25-500, 25-250, 25-100, 25-75, 25-50, 50-1000, 50-750, 50-500, 50-250, 50-100, 100-1000, 100-750, 100-500, 100-250, 250-1000, 250-750, 250-500, 500-1000, 500-750, or 750-1000, different RNA. In some embodiments, the composition comprises more than 1000 different RNA.

In some embodiments, a composition comprising two or more different RNA binding proteins and one or more RNA molecules is disclosed. In some embodiments, one or more of the RNA molecules in the composition are bound a RNA binding protein. In some embodiments, at least 1, 2, 3, 4, 5, 6, 7, 8, 9, or 10, different RNA in the sample are bound to a single RNA binding protein, or a number of RNA that is in a range defined by any two of the preceding values are bound to a RNA binding protein. For example, in some embodiments, between 1-10, 1-8, 1-6, 1-4, 1-2, 2-10, 2-8, 2-6, 2-4, 4-10, 4-8, 4-6, 6-10, 6-8, or 8-10, different RNAs are bound to each different RNA binding protein.

In some embodiments, the methods and compositions disclosed herein are used to identify one or more RBP: RNA interactions. In some embodiments, the methods and compositions disclosed herein are used to identify 1, 2, 3, 4, 5, 6, 7, 8, 9, 10, 20, 25, 30, 40, 50, 60, 70, 75, 80, 90,100,200, 250, 300, 400, 500, 600, 700, 750, 800, 900, or 1000, different RBP: RNA interactions or to identify a number of RBP: RNA interactions that is in a range defined by any two of the preceding values. For example, in some embodiments, between 1-1000, 1-750, 1-500, 1-250, 1-100, 1-75, 1-50, 1-25, 1-10, 1-5, 5-1000, 5-750, 5-500, 5-250, 5-100, 5-75, 5-50, 5-25, 5-10, 10-1000, 10-750, 10-500, 10-250, 10-100, 10-75, 10-50, 10-25, 25-1000, 25-750, 25-500, 25-250, 25-100, 25-75, 25-50, 50-1000, 50-750, 50-500, 50-250, 50-100, 100-1000, 100-750, 100-500, 100-250, 250-1000, 250-750, 250-500, 500-1000, 500-750, or 750-1000, RBP: RNA interactions are identified. In some embodiments, more than 1000 RBP: RNA interactions are identified.

Some aspects of the present disclosure are directed to kits for identifying interactions between RBP and RNA. In some embodiments, the kit comprises: an antibody-bead conjugate pool. In some embodiments, the antibody-bead conjugate pool comprises a plurality of antibody-bead conjugate populations. Each antibody-bead conjugate population in the antibody-bead conjugate pool comprises a different antibody identifying oligonucleotide. In some embodiments, the antibody-identifying oligonucleotide is attached to the bead of each antibody-bead conjugate. In some embodiments, each antibody-bead conjugate population in the antibody-bead conjugate pool is specific for a different RNA binding protein. Optionally, in some embodiments, the kits of the present disclosure further comprise a cross-linking agent. In some embodiments, the kit comprises a biotinylated protein G bead bound to a streptavidin-biotin-tag complex. In some embodiments, the kit comprises one or more barcode oligonucleotides.

In some embodiments, the kit's crosslinking agent comprises an amine-to-amine crosslinker (such as disuccinimidyl suberate or disuccinimidyl tartrate), or a sulfhydryl-to-sulfhydryl crosslinker (such as bis-maleimidoethane or dithio-bis-maleimidoethane), or an aryl-azide (such as N-5-Azido-2-nitrobenzyloxysuccinimide or sulfosuccinimidyl 6-(4′-azido-2′-nitrophenylamino) hexanoate), or a diazirine (such as succinimidyl 4,4′-azipentanoate). In the kit of some embodiments, the crosslinking agent comprises an agent selected from the group consisting of an NHS ester, an imidoester, a difluoro group, an NHS-haloacetyl group, an NHS-maleimide group, an NHS-pyridyldithiol group, a carbodiimide ester and NHS ester, a malemide and a hydrazine group, a pyridyldithiol and a hydrazine group, a NHS ester and an aryl azide, a NHS ester and a diazirine, a NHS ester and an aryl azide, and a diazirine.

In some embodiments, the kit comprises multiple unique barcode oligonucleotides. In some embodiments, the kit comprises 1, 2, 3, 4, 5, 6, 7, 8, 9, 10, 20, 25, 30, 40, 50, 60, 70, 75, 80, 90, or 100 unique barcode oligonucleotides, or a number of unique barcode oligonucleotides that is in a range defined by any two of the preceding values. For example, in some embodiments, the kit comprises between 1-100, 1-75, 1-50, 1-25, 1-10, 1-5, 5-100, 5-75, 5-50, 5-25, 5-10, 10-100, 10-75, 10-50, 10-25, 25-100, 25-75, 25-50, 50-100, 50-75, or 75-100, unique barcode oligonucleotides.

### Numbered Arrangements

Some embodiments provided herein are described by way of the following provided numbered arrangements and also provided as possible combinations or overlapping embodiments:
1. A method of detecting an association between a RNA binding protein and a RNA, the method comprising:
   a. providing an antibody-bead conjugate pool comprising a plurality of antibody-bead conjugate populations, wherein
      each antibody-bead conjugate population comprises a plurality of antibody-bead conjugates, wherein
      each antibody-bead conjugate in an antibody-bead conjugate population comprises an antibody specific for the same RNA binding protein and an RBP-identifying oligonucleotide, wherein
      each antibody-bead conjugate population in the antibody-conjugate pool comprises antibodies specific for a different RNA binding protein;
   providing a composition comprising a plurality of crosslinked RNA: RBP complexes,
   immunopurifying the cross-linked RNA using the antibody-bead conjugate pool,
   performing one or more rounds of split-and-pool barcoding, wherein
   b. during each of the one or more rounds of split-and-pool barcoding the same barcode oligonucleotide is added to both the RBP identifying oligonucleotide and the immunopurified RNA on each antibody-bead conjugate;
   sequencing the barcoded molecules, and
   assigning the one or more RNA molecules to their corresponding RNA binding protein.
2. The method of arrangement 1, wherein the antibody-bead conjugate pool comprises a biotinylated protein G bead bound to a streptavidin-biotin-tag complex.
3. The method of arrangement 1, wherein generating an antibody-bead conjugate pool comprises:
   a. incubating one or more populations of biotinylated protein G beads with a streptavidin-biotin oligo complex, wherein
      each population of beads is labeled with an antibody identifying oligonucleotide;
   b. incubating each of the one or more populations of beads with an antibody, wherein
      each population of beads is incubated with a different antibody; and
   c. combining each of the one or more populations of beads to generate an antibody-bead conjugate pool.
4. The method of arrangement 1, wherein the bead comprises a protein A, protein G, or protein A/G bead.
5. The method of arrangement 1, wherein the composition comprises a cell.
6. The method of arrangement 6, further comprising lysing the cell.
7. The method of arrangement 1, wherein the composition comprises a cell lysate.
8. The method of arrangement 1, wherein providing a composition comprising crosslinked RNA and RNA binding proteins comprises applying a crosslinking agent to a composition comprising a plurality of RNA molecules and a plurality of RNA binding proteins.
9. The method of arrangement 1, wherein assigning the one or RNA to their corresponding RNA binding protein comprises matching the antibody-bead conjugate and RNA based on their shared barcode oligonucleotides.
10. The method of arrangement 1, wherein immunopurifying the cross-linked RNA using the antibody-bead conjugate pool enriches the RNA binding protein by at least about 2-fold relative to a negative control.
11. The method of arrangement 1, wherein up to 10 rounds of split-and-pool barcoding are performed.
12. The method of arrangement 1, wherein up to unique 20 barcodes are used in each round of split-and-pool barcoding.
13. The method of arrangement 1, wherein at least 10 different RBP: RNA interactions are identified.
14. An antibody-bead conjugate population, the antibody-bead conjugate population comprising a plurality of antibody-bead conjugates, wherein
   each bead in the antibody-bead conjugate population is conjugated to an antibody or binding fragment thereof specific for a RNA binding protein, wherein
      each antibody or binding fragment thereof in the antibody-bead conjugate population is specific for the same RNA binding protein;
   each bead in the antibody-bead conjugate population is labeled with the same RNA binding protein-identifying oligonucleotide, wherein
15. The method of arrangement 24, wherein the bead comprises a protein A, protein G, or protein A/G bead.
16. The method of arrangement 24, wherein the bead is a magnetic bead.
17. An antibody-bead conjugate pool, the antibody-bead conjugate pool comprising a plurality of antibody-bead conjugate populations, wherein
   each antibody-bead conjugate population comprises a plurality of antibody-bead conjugates comprising an antibody or binding fragment thereof specific for a single RNA binding protein and an RBP-identifying oligonucleotide, and wherein
   each antibody-bead conjugate population in the antibody-conjugate pool is specific for a different RNA binding protein.
18. A kit comprising:
   an antibody-bead conjugate pool, wherein
      the antibody-bead conjugate pool comprises a plurality of antibody-bead conjugate populations, wherein
      each antibody-bead conjugate population in the antibody-bead conjugate pool comprises a different antibody identifying oligonucleotide, wherein
         the barcode is attached to the bead of each antibody-bead conjugate, and
      each antibody-bead conjugate population in the antibody-bead conjugate pool is specific for a different RNA binding protein.
19. The kit of arrangement 28, wherein the antibody-bead conjugate pool comprises a biotinylated protein G bead bound to a streptavidin-biotin-tag complex.
20. The kit of arrangement 28, further comprising one or more barcode oligonucleotides.
21. The kit of arrangement 28, wherein the kit comprises up to 100 unique barcode oligonucleotides.
22. The kit of arrangement 28, further comprising a cross-linking agent.

### EXAMPLES

The results disclosed herein demonstrate that SPIDR can accurately map numerous RBPs within a single experiment. The number of antibodies used in the examples provided herein merely reflect the availability of high-quality antibodies. As such, it is expected that one skilled in the art would understand that the approaches disclosed herein can readily be applied to hundreds or thousands of proteins simultaneously. Because of this, SPIDR represents a critical technology for exploring the many thousands of human proteins that have been reported as putative RNA binding proteins but that remain largely uncharacterized. For example, in some embodiments, the disclosure herein will be used to assess the putative functions of one or more, if not all, of the >20,000 annotated ncRNAs which have remained largely uncharacterized.

Because the number of cells required to perform SPIDR is comparable to that of a traditional CLIP experiment, yet a single SPIDR experiment reports on the binding behavior of numerous RBPs, this approach dramatically reduces the number of cells required to map an individual RBP. Accordingly, SPIDR is a valuable tool for studying RBP-RNA interactions in many different contexts, including within rare cell types and patient samples where large numbers of cells may be difficult to obtain.

The results disclosed herein demonstrate that SPIDR generates single nucleotide contact maps that accurately recapitulate the RNA-protein contacts observed within structural models. SPIDR's simultaneous targeting of all proteins within a complex adds high-resolution binding information for entire RNP complexes in a single experiment. In conjunction with more traditional structural biology methods, this approach will help elucidate the precise structure of various RNP complexes, including for mapping proteins that are not currently resolved within these structures (e.g. LARP1 binding within the 48S ribosome).

In addition to accurately measuring multiple proteins simultaneously, because of the nature of the split-and-pool barcoding strategy used, the approaches disclosed herein also allow for multiple samples to be pooled within a single experiment. This ability to simultaneously map multiple proteins across different samples and conditions will enable exploration of RBP binding patterns and their changes across diverse biological processes and disease states. Until now, systematic comparative studies of RBP-RNA interaction changes at scale have been impossible, even for large consortia (e.g., ENCODE), which have invested massive amounts of time and effort to generate CLIP-seq data for only two cell lines. The results presented in the Examples and elsewhere throughout the present disclosure highlight the critical value of SPIDR for enabling exploration of RBP dynamics across samples. Specifically, RBPs was not commonly thought to directly bind to mRNA, nonetheless, including 4EBP1 within the larger pool of target proteins allowed the discovery of changes across two different experimental conditions that may explain how specificity of mTOR-mediated translational suppression is achieved.

Although differential RNA binding properties of 4EBP1/LARP1 were the focus of some examples disclosed herein, there are many additional insights into RBP biology that can be uncovered using the methods and compositions disclosed herein. In some embodiments, RBPs of great interest, for example because of a known link between the RBP and a disorder, for example, but not limited, to a neurodegenerative disorder such as amyotrophic lateral sclerosis (ALS), may be examined. These observations could provide new mechanistic insights into how disruption of this RBP impacts the disease state, such as splicing changes and pathogenesis in neurodegeneration.

SPIDR, was used to explore changes in RBP binding upon mTOR inhibition. SPIDR identified that 4EBP1 acts as a dynamic RBP that selectively binds to 5′-untranslated regions of specific translationally repressed mRNAs only upon mTOR inhibition. This observation provides a potential mechanism to explain the specificity of translational regulation controlled by mTOR signaling.

### Example 1: A Highly Multiplexed Method for Mapping RBP-RNA Interactions

SPIDR was developed to enable highly multiplexed mapping of RBPs to individual RNAs transcriptome-wide.

FIG. 1A is a schematic overview of some embodiments of the Split and Pool Identification of RBP targets (SPIDR) method.

FIG. 7 schematically depicts some embodiments for a multiplexed antibody-bead labeling strategy. Populations of biotinylated protein G beads are incubated with a streptavidin-biotin oligo complex. Each population of beads is labeled with an oligo with a specific sequence and then incubated with one type of capture antibody such that each population has a unique capture antibody and a corresponding oligo tag that can be recognized after sequencing. Populations are combined to create the bead pool.

On-bead immunoprecipitation (IP) of RBPs in UV-crosslinked lysates was performed using standard conditions and individual protein identities were assigned to their associated RNAs using split-and-pool barcoding, where the same barcode strings were added to both the oligonucleotide bead tag and immunopurified RNA (FIG. 1A). The split-and-pool tagging method was dramatically simplified to allow for performance of the entire protocol in ˜1 hour without the need for specialized equipment (see Materials and Methods, below).

After split-and-pool tagging and subsequent library preparation, all barcoded DNA molecules (antibody-bead tags and the converted cDNA of RNAs bound to corresponding RBPs) were sequenced. All antibody-bead tags and RNA reads were then matched by their shared barcodes; these are referred to herein as “SPIDR clusters” (FIG. 1A). All SPIDR clusters were merged by protein identity (specified by the antibody-bead tag) to generate a high-depth binding map for each protein. The resulting datasets were analogous to those generated by traditional individual CLIP approaches.

To ensure that IP using a pool containing multiple antibodies could successfully and specifically purify each of the individual proteins, an IP in K562 cells was performed using a pool of antibodies against 39 RBPs.

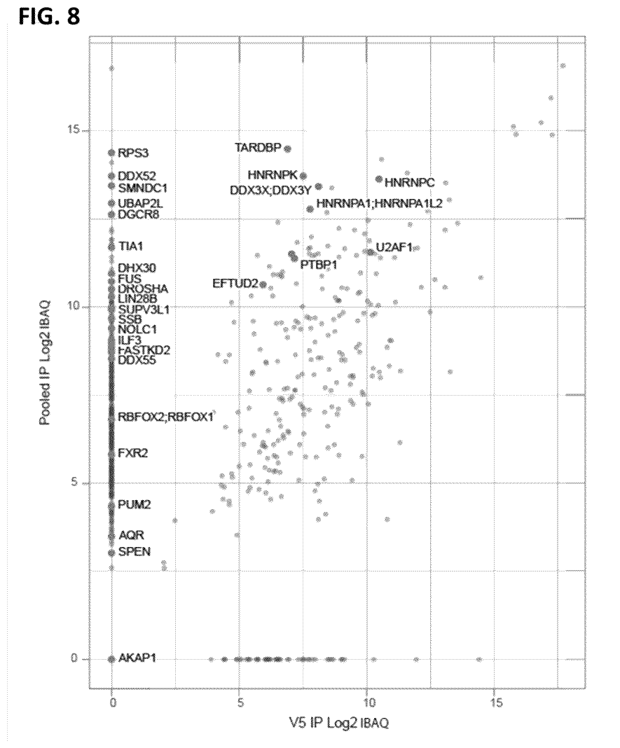

FIG. 8 depicts some embodiments of a scatter plot showing log 2 transformed IBAQ (intensity based absolute quantification) values for all identified proteins in either the pooled IP with 39 targets (y-axis) versus those detected with a V5 negative control IP (x-axis) by LC-MS/MS. Target proteins that should be detected by the antibodies included in the pool of 39 used are marked in dark gray.

The purified proteins were measured by liquid chromatography tandem mass spectrometry (LC-MS/MS). 35 of the 39 targeted RBPs enriched at least 2-fold relative to a negative control, showing that multiplexed enrichment of several RBPs simultaneously is possible (FIG. 8). The few exceptions were RBPs that were simply not detected (neither in the pooled IP nor under control conditions) and likely reflect either a poor antibody or lack of RBP expression in this cell line.

SPIDR Accurately Maps Dozens of RBPs within a Single Experiment

SPIDR was performed in two widely studied human cell lines (K562 and HEK293T cells) to test whether SPIDR accurately maps RBPs to RNA.

FIG. 1B is a schematic list of some embodiments of different RBPs mapped by SPIDR in K562 and/or HEK293T cells, functional assignments based on literature review.

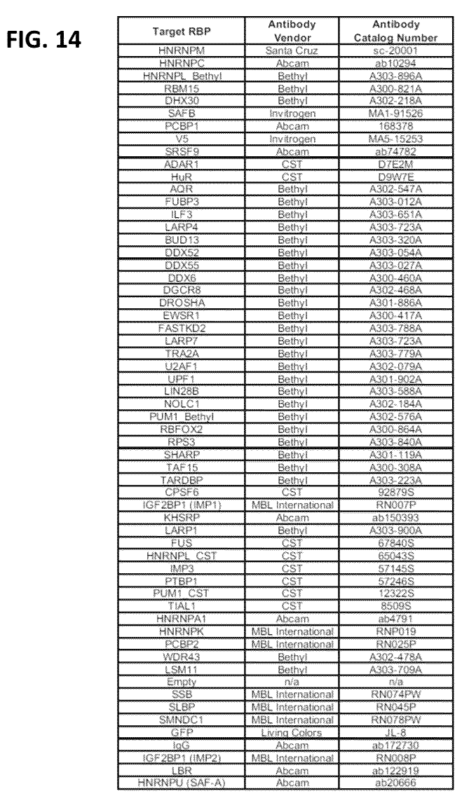

FIG. 14 is a table depicting some embodiments of an overview of the SPIDR experiment in K562 cells. The protein targets are listed, as well as the vendors and product numbers of the corresponding antibodies.

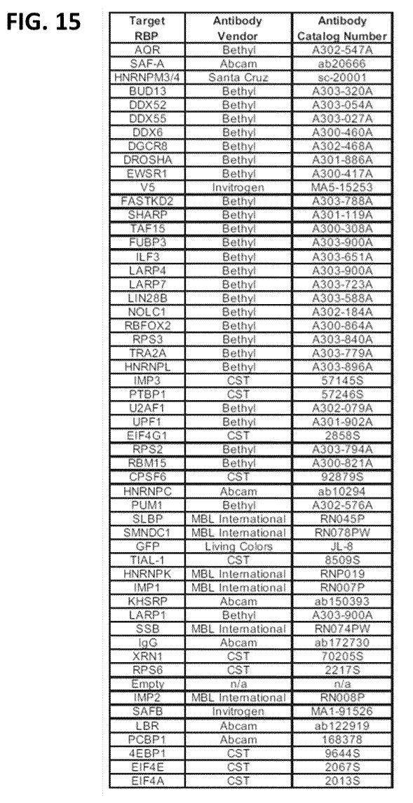

FIG. 15 is a table depicting some embodiments of an overview of the SPIDR experiment HEK293T cells treated with Torin or Control (solvent only). The protein targets are listed, as well as the vendors and product numbers of the corresponding antibodies.

Antibody bead pools containing 68 uniquely tagged antibody-beads targeting 62 distinct RBPs across the RNA life cycle, including splicing, processing, and translation factors (FIG. 1B, FIG. 14, FIG. 15) were generated.

Antibodies against epitopes not present in endogenous human cells (GFP and V5), antibodies that lack affinity to any epitope (mouse IgG), and oligonucleotide-labeled beads lacking any antibody (empty beads) were included as negative controls.

Using these pools, SPIDR was performed on 10 million UV-crosslinked cells.

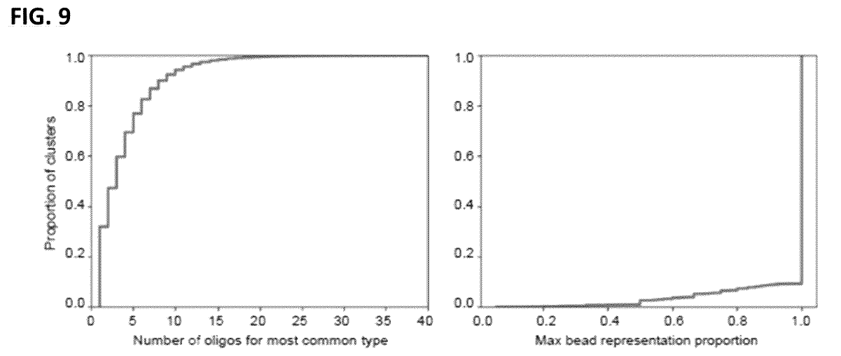

FIG. 9 depicts some embodiments of observed distributions of labeled beads after sequencing. Each bead is defined in sequencing by a particular, unique combinatorial barcode acquired during split-pool. A SPIDR cluster represents any set of molecules, oligo or RNA, that share the same bead combinatorial barcode. Left: CDF plot showing some embodiments of the number of independent oligos matched within an individual SPIDR cluster. Right: CDF plot describing some embodiments of the degree of heterogeneity of these detected oligos within each SPIDR cluster, as determined by oligos with a shared combinatorial barcode. X axis represents the homogeneity of the oligo types with 1 indicating that all oligos are of the same type.

Focusing on the K562 data (which were sequenced at greater depth), a median of 4 oligonucleotide tags per SPIDR cluster were generated with the majority of clusters (>80%) containing tags representing only a single antibody type (FIG. 9), indicating that there is minimal ‘crosstalk’ between beads in a SPIDR experiment. This specificity enables unique assignment of RNA molecules to their corresponding RBPs. After removing PCR duplicates, each sequenced RNA read was assigned to its associated RBP.

FIG. 1C is an example of some embodiments of a raw alignment data for a pool (all reads before splitting by bead identities) and for specific RBPs (all reads assigned to specific RBP beads) across the XIST RNA. Blocks represent exons, lines introns, and thick blocks are the annotated XIST repeat regions (A-E).

FIG. 1D depicts some embodiments of a raw alignment data for some SLBP across the H3C2 histone mRNA. Top track depicts some embodiments of pooled alignment data; tracks below depict some embodiments of reads assigned to SLBP or other RBPs and controls.

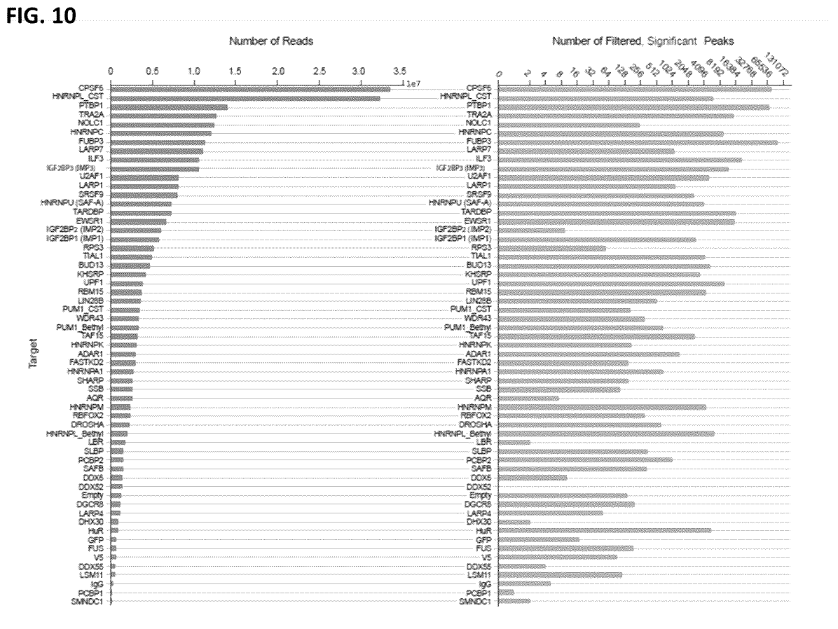

FIG. 10 depicts some embodiments of the number of deduplicated mapped reads and number of significant binding sites within uniquely mapped genomic regions per IP. The order is determined by the number of unique mapped reads in both plots.

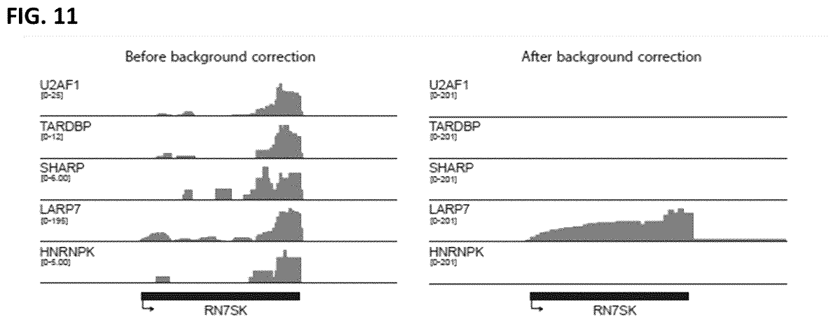

FIG. 11 depicts some embodiments of an example of a background correction method that utilizes the total read coverage across all proteins to normalize each individual protein. Shown are example tracks on RN7SK before and after background correction. Left: Raw alignment data for the entire pooled dataset (top track) and for representative antibodies against U2AF1, TARDBP, SHARP, LARP7 and HNRNPK on RN7SK. Right: Background corrected data for the same set of antibodies. Signal that was not antibody-specific is normalized out. The reads in the right are binned in 5 nucleotide windows. RN7SK is known to be bound by LARP7.

High confidence binding sites were identified by comparing read coverage across an RNA to the coverage in all other targets in the pooled IP (FIG. 10, FIG. 11; see Materials and Methods, below, for details). Using this approach, the precise binding sites for SAF-A, PTBP1, SPEN, and HNRNPK on the XIST RNA 17,20,23 were determined (FIG. 1C). Although most proteins (38/53 RBPs in K562) contained more than 2 million mapped RNA reads (FIG. 10), specific binding to known target sites was observed even for RBPs with lower numbers of reads. For example, SLBP (Stem Loop Binding Protein) had only 1.5 million mapped reads yet displayed strong enrichment specifically at the 3′ ends of histone mRNAs as expected29 (FIG. 1D).

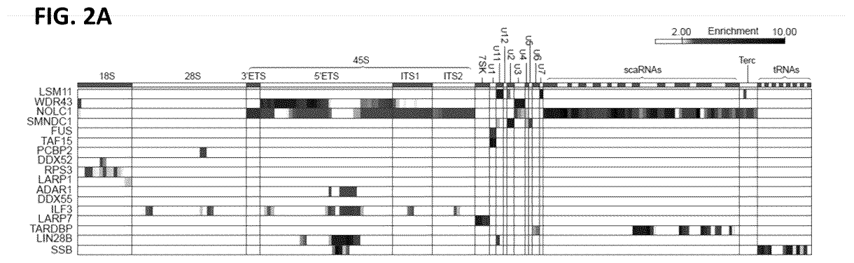

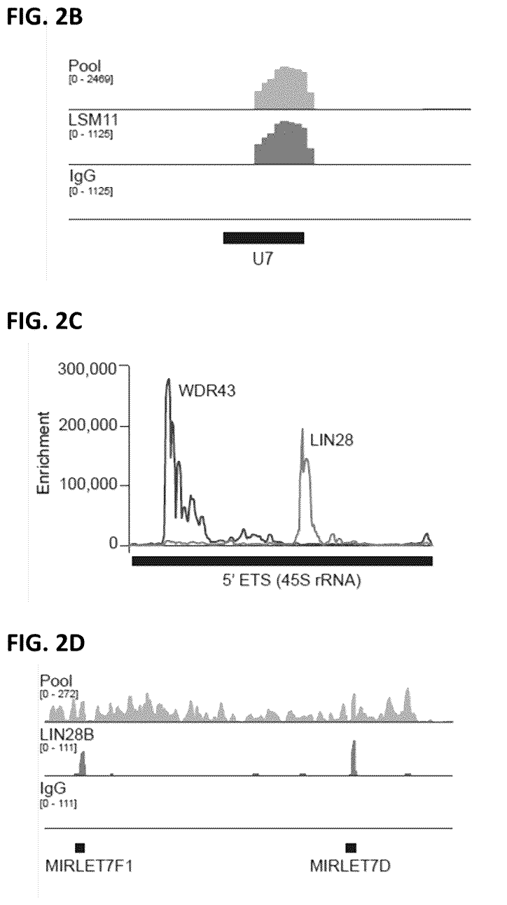

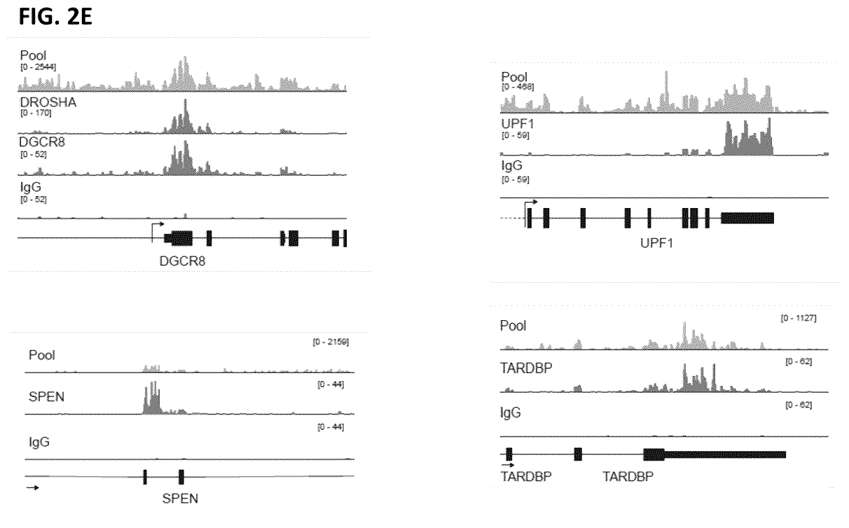

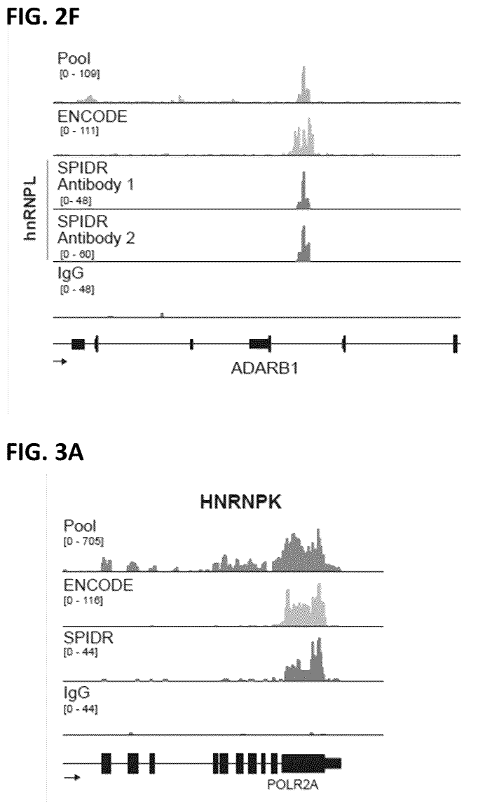

FIG. 2A depicts some embodiments of RNA binding patterns of selected RBPs (rows) relative to 100 nt windows across each classical non-coding RNA (columns). Each bin is colored based on the enrichment of read coverage per RBP relative to background.

FIG. 2B depicts some embodiments of sequence read coverage for LSM11 binding to U7 snRNA. For all tracks, “pool” refers to all reads prior to splitting them by paired barcodes (shown in light gray), and individual tracks (shown in dark gray) reflect reads after assignment to specific antibodies.

FIG. 2C depicts some embodiment of enrichment of read coverage relative to background for WDR43 and LIN28B over the 5′ ETS region of 45S RNA.

FIG. 2D depicts some embodiments of sequence reads coverage for LIN28B binding to let-7 miRNAs.

FIG. 11 depicts some embodiments of an example of a background correction method that utilizes the total read coverage across all proteins to normalize each individual protein. Shown are example tracks on RN7SK before and after background correction. Left: Raw alignment data for the entire pooled dataset (top track) and for representative antibodies against U2AF1, TARDBP, SHARP, LARP7 and HNRNPK on RN7SK. Right: Background corrected data for the same set of antibodies. Signal that was not antibody-specific is normalized out. The reads in the right are binned in 5 nucleotide windows. RN7SK is known to be bound by LARP7.

The quality, accuracy, and resolution of the SPIDR binding maps and the scope of the SPIDR method was assessed:
(i) Accurate mapping of classical RNPs. RBPs of diverse functionality, such as those which bind preferentially to RNAs coding for proteins and/or lncRNAs, to introns, exons, miRNAs, etc., as well as more “classical” ribonuclear protein (RNP) complexes, such as the ribosome or spliceosome, were targeted (FIG. 2A). Precise binding to the expected RNAs and binding sites was observed. For example, binding of each of the following was observed: LSM11 to the U7 small nuclear RNA (snRNA) 33 and the telomerase RNA component (TERC) 34 (FIG. 2A and FIG. 2B); WDR43, a protein that is involved in ribosomal RNA (rRNA) processing, to the 45S pre-rRNA and the U3 small nucleolar RNA (snoRNA), which is involved in rRNA modification35 (FIG. 2A and FIG. 2C); LIN28B to a distinct region of the 45S pre-rRNA, consistent with recent reports of its role in ribosomal RNA biogenesis in the nucleolus36 (FIG. 2A and FIG. 2C); NOLC1 (also known as NOPP140), a protein that localizes within the nucleolus and Cajal bodies37,38, to both the 45S pre-rRNA (enriched within the nucleolus) and various small Cajal-body associated RNAs (scaRNAs) (FIG. 2A); DDX52, a DEAD-box protein that is predicted to be involved in the maturation of the small ribosomal subunit39,40 and RPS3, a structural protein contained within the small ribosomal RNA subunit, to distinct sites on the 18S rRNA (FIG. 2A); FUS and TAF15 to distinct locations on the U1 snRNA41,42 (FIG. 2A); SMNDCI specifically to the U2 snRNA43 (FIG. 2A); SSB (also known as La protein) binding to tRNA precursors consistent with its known role in the biogenesis of RNA Polymerase III transcripts44,45 (FIG. 2A); LIN28B to the let-7 miRNA46-50 (FIG. 2D); and LARP7 binding to 7SK51 (FIG. 2A, FIG. 11).

FIG. 2E depicts some embodiments of sequence reads coverage for DROSHA/DGCR8, UPF1, SPEN, and TARDBP to their respective mRNAs.

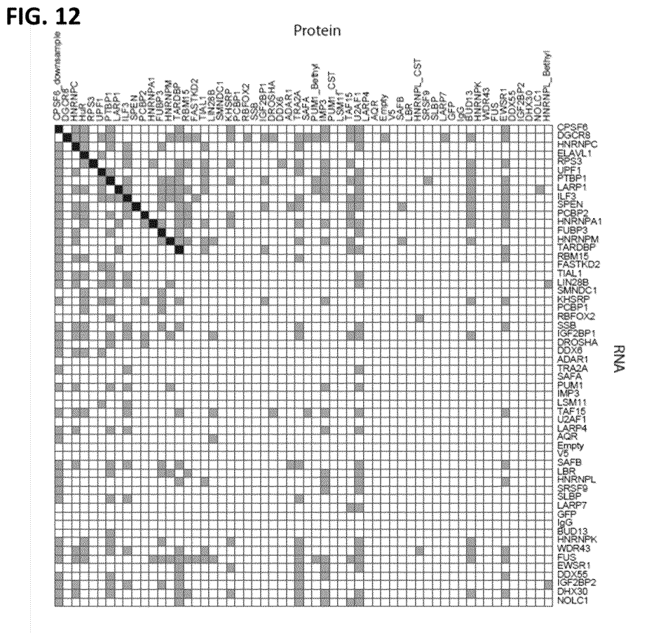

FIG. 12 depicts some embodiments of an auto-regulatory binding matrix with protein (x-axis) binding to each mRNA (y-axis). Each target protein included in SPIDR performed in K562 cells marked by whether it has significantly enriched binding within its own RNA, or in any of the other SPIDR target RNAs. Proteins that bind their own RNA are marked in black, instances of binding to genes of other SPIDR targets are marked in gray.
(ii) Many RBPs bind their own mRNAs to autoregulate expression levels. Many RBPs have been reported to bind their own mRNAs to control their overall protein levels through post-transcriptional regulatory feedback. For example, SPEN protein binds its own mRNA to suppress its transcription, UPF1 binds its mRNA to target it for Nonsense Mediated Decay56, TARDBP binds its own 3′-UTR to trigger an alternative splicing event that results in degradation of its own mRNA, and DGCR8, which together with DROSHA forms the known microprocessor complex, binds a hairpin structure in DGCR8 mRNA to induce cleavage and destabilization of the mRNA (FIG. 2E). In addition to these cases, autoregulatory binding of proteins to their own mRNAs was observed for nearly a third of the targeted RBPs (15 proteins) (FIG. 12).

FIG. 2F depicts some embodiments of sequence reads coverage for two distinct antibodies to HNRNPL in a single SPIDR experiment. For comparison, HNRNPL coverage from the ENCODE-generated eCLIP data is also shown.
(iii) Different antibodies that capture the same protein or multiple proteins within the same complex show similar binding. The possibility that including antibodies against multiple proteins contained within the same complex, or that otherwise bind to the same RNA, within the same pooled sample could compete against each other and therefore limit the utility of large-scale multiplexing was considered. However, this was not observed to be the case; in fact, antibodies against different proteins known to occupy the same complex displayed highly comparable binding sites on the same RNAs. For example, DROSHA and DGCR8, two proteins that bind as part of the microprocessor complex, showed highly consistent binding patterns across known miRNA precursors with significant overlap in their binding sites (odds-ratio of 316-fold, hypergeometric p-value <10-100). Similarly, when two distinct antibodies targeting the same protein were included, HNRNPL, highly comparable binding profiles for both antibodies (FIG. 2F) and significant overlap in defined binding sites (odds-ratio of 15-fold, hypergeometric p-value <10-100) was observed. Taken together, these results indicate that SPIDR can be used to map different RBPs that bind to the same RNA targets and can successfully map multiple antibodies targeting the same protein. As such, SPIDR may be a particularly useful tool for directly screening multiple antibodies targeting the same protein to evaluate utility for use in CLIP-like studies.

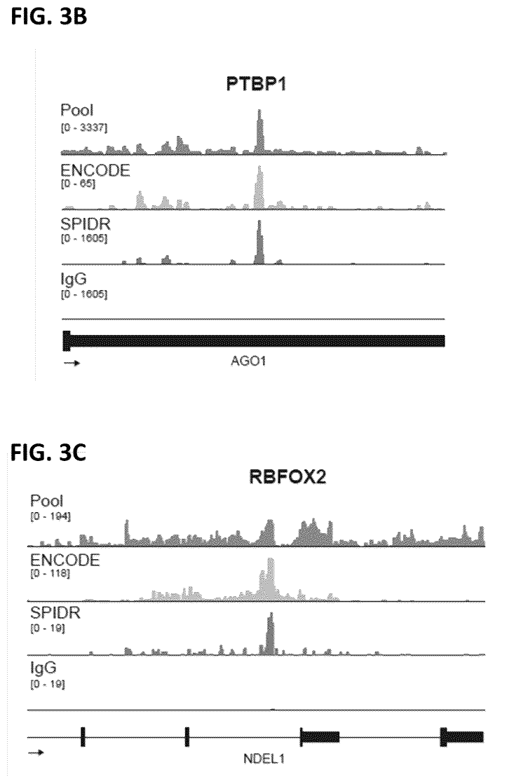

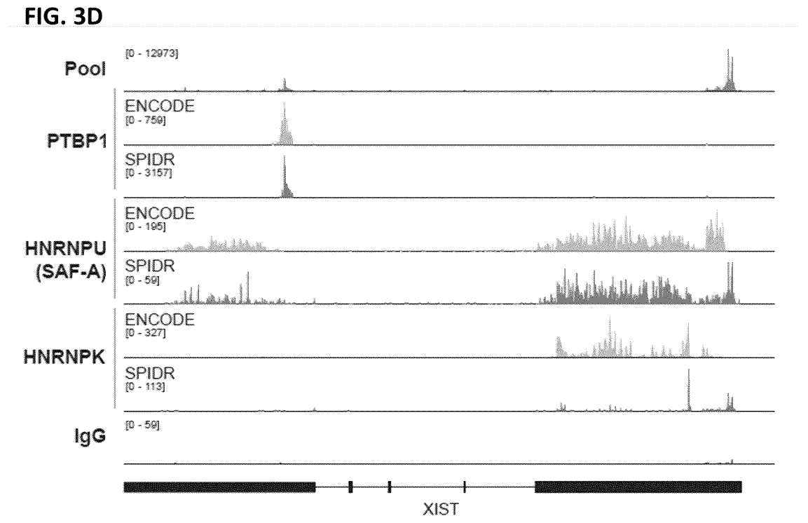

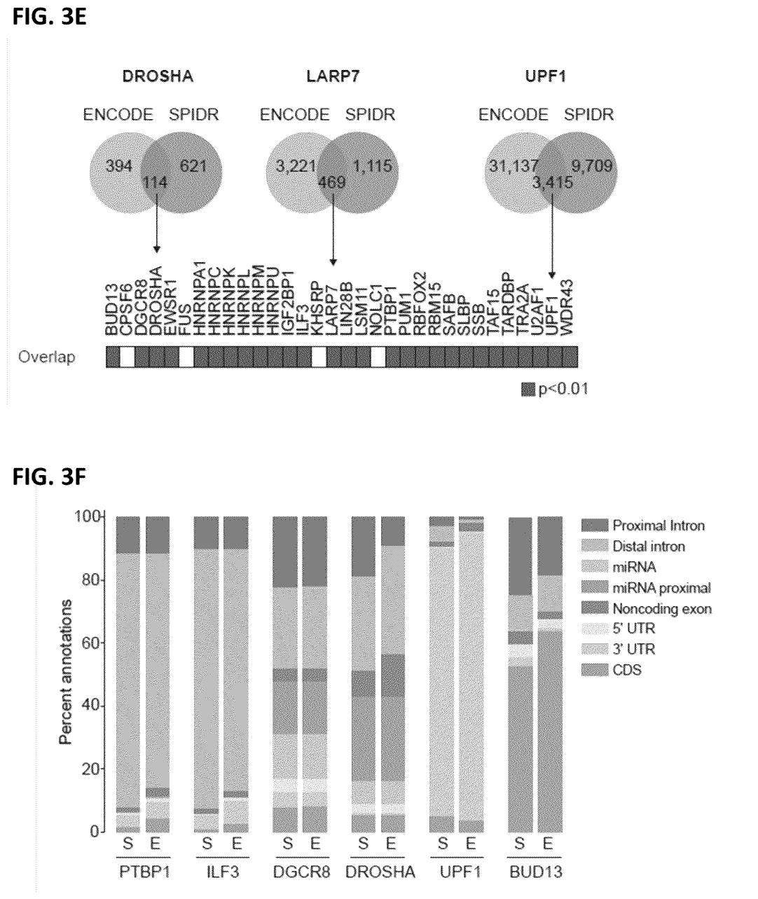

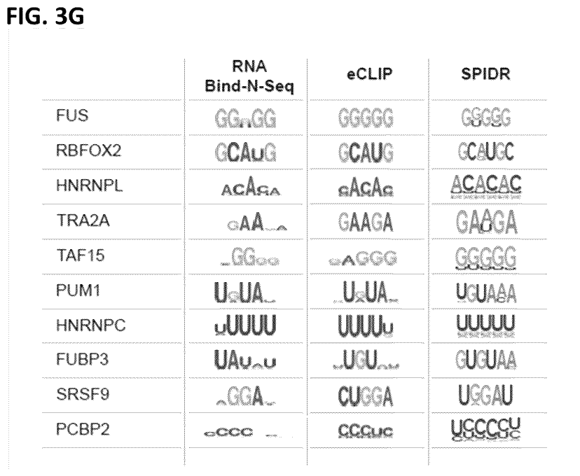

FIG. 3A-G depicts some embodiments of examples of concordant binding identified by eCLIP (ENCODE consortium) and SPIDR. Sequence reads coverage is shown for individual proteins measured by ENCODE and SPIDR along with a negative control (IgG). FIG. 3A depicts some embodiments of concordant binding of HNRNPK. FIG. 3B discloses some embodiments of concordant binding of PTBP1. FIG. 3C depicts some embodiments of concordant binding of RBFOX2. FIG. 3D depicts some embodiments of a comparison of ENCODE and SPIDR data for multiple proteins bound to the XIST lncRNA. Sequence reads coverage for PTBP1, HNRNPU (SAF-A), and HNRNPK are shown. FIG. 3E depicts some embodiments of the significance of overlap between binding sites detected by SPIDR and those identified within paired proteins in the ENCODE data. Each bin represents the paired protein between both experiments, blue represent a hypergeometric p-value of less than 0.01. FIG. 3F depicts some embodiments of peak annotation in matched SPIDR and ENCODE data. Stacked bar plot showing the percentage of peaks detected in the SPIDR(S) or ENCODE (E) datasets in various annotation categories. FIG. 3G depicts some embodiments of a comparison of significant motifs identified within SPIDR peaks (right, p-value threshold <1e-40) to those reported for RNA Bind-n-Seq (left) or eCLIP (middle).

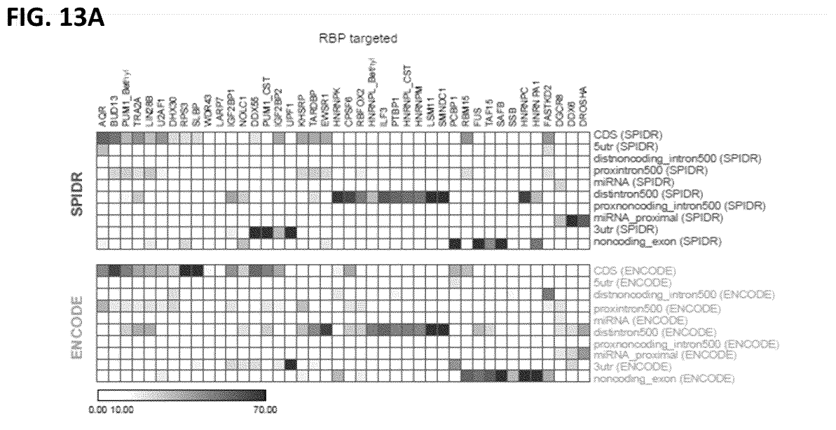

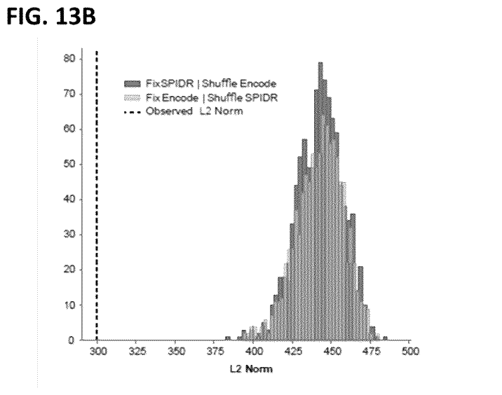

FIG. 13A depicts some embodiments of heatmaps showing the percentage of significant binding sites in each of the annotation categories for SPIDR performed in K562 cells and ENCODE.

FIG. 13B depicts some embodiments of a quantitative assessment of the similarity of heatmaps between SPIDR and ENCODE. The Euclidean distance (L2 norm) between the ENCODE and SPIDR percentage tables/heatmaps is calculated. The calculated distance is indicated by the dashed line. The statistical significance is calculated by randomly shuffling the columns of either the SPIDR percentage table and keeping the original ENCODE table or vice versa, meaning shuffling the columns of the ENCODE table and keeping the original SPIDR table. This is done 1000 times in each direction and every time the Euclidean distance was calculated. The values are represented by the two histograms. The Euclidean distance of all of the randomly shuffled 2000 comparison is always larger than of the true pair, which shows that the two original annotation tables from SPIDR and ENCODE are highly significantly similar (p-value <0.0005).
(iv) Transcriptome-wide SPIDR maps are highly comparable with CLIP. Because K562 represents the ENCODE-mapped cell line with the largest number of eCLIP datasets, we were able to benchmark our SPIDR results directly to those generated by ENCODE. To do this, we compared the profiles for each of the 33 RBPs that overlap between SPIDR and ENCODE datasets in K562 cells23,28,29 (see Materials and Methods, below). Highly overlapping binding patterns for most RBPs were observed, including: HNRNPK binding to POLR2A (FIG. 3A), PTBP1 binding to AGO1 (FIG. 3B), RBFOX2 to NDELI (FIG. 3C) and the binding of several known nuclear RBPs to XIST (FIG. 3D). To explore this data on a global scale, we compared RNA binding sites for each RBP and observed significant overlap between SPIDR- and ENCODE-derived binding sites for the vast majority of proteins (29/33, p<0.01, FIG. 3E). Moreover, we observed that in virtually all cases each RBP preferentially binds to the same RNA features (e.g., introns, exons, CDS, miRNAs, 5′ and 3′UTRs) in both datasets (FIG. 3F, FIG. 13A-B). Finally, the binding motifs identified within the significant SPIDR-defined binding sites match those defined by CLIP and in vitro binding assays29 (e.g., RNA Bind-N-Seq, FIG. 3G).

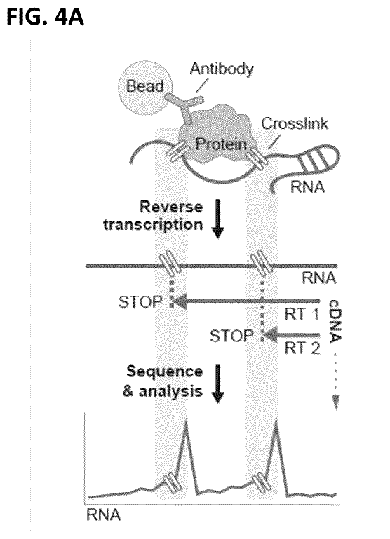

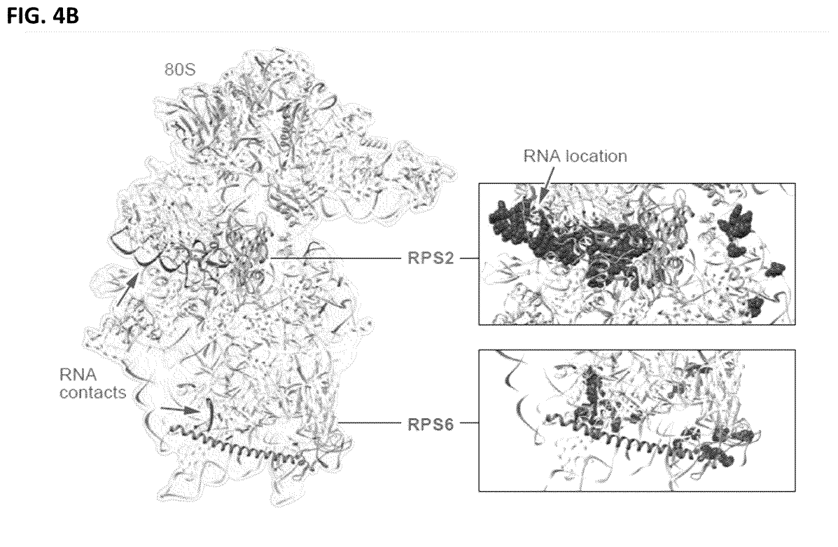

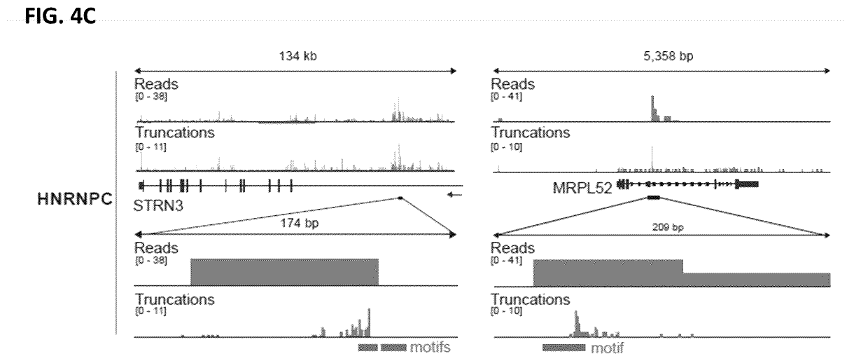

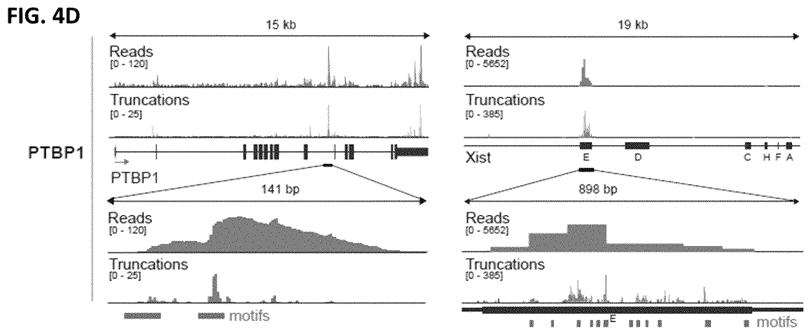

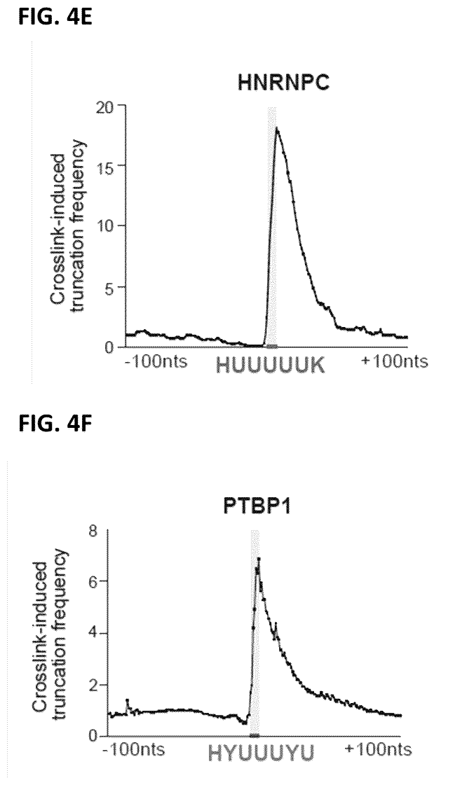

FIG. 4A is a schematic showing how reverse transcription pause sites can be used to map RBP-RNA interactions at single nucleotide resolution. UV light crosslinks the RBP to the target RNA at points of direct contact. During reverse transcription, the enzyme preferentially stalls at the crosslinking site, leading to termination of cDNA synthesis (STOP). Mapping the 3′-end of the cDNA (truncations) may identify the RBP binding site at single nucleotide resolution.

FIG. 4B depicts some embodiments of the SPIDR determined binding sites of RPS2 and RPS6 overlayed on the known 80S ribosome structure. SPIDR data shown is from HEK293T cells.

FIG. 4C depicts some embodiments of HNRNPC binding sites for STRN3 (left) and MRPL52 (right). Both raw read alignments (“Reads”, top) and 3′-end truncations of the cDNA (“Truncations”, bottom) are shown. The upper two panels show the mapped reads and truncations for the whole gene, the lower two panels are zoomed-in on the indicated region.

FIG. 4F depicts some embodiments of the truncation frequency (3′ ends of the mapped cDNA reads) over all significantly enriched PTBP1 peaks. Truncation frequency is shown relative to the motif position within each peak. The region of the steep frequency rise of truncations is shown by the line on the y-axis and corresponds to the displayed sequence.
(v) SPIDR enables high-resolution RBP mapping at single nucleotide resolution. Next, we explored whether SPIDR can provide single nucleotide resolution maps of precise RBP-RNA binding sites, as is the case for some current CLIP-seq approaches. Specifically, UV crosslinking creates a covalent adduct at the site of RBP-RNA crosslinking, which leads to a preferential drop-off of the reverse transcriptase at these sites (FIG. 4A). To explore this, we computed the number of reads that end at each position of an RNA (truncations) and compared these counts to those expected by chance. We observed strong positional enrichments at known protein binding sites. For example, we observe strong enrichment for RPS2 and RPS6-two distinct structural components of the small ribosomal RNA subunit—at the precise locations where these proteins are known to contact the 18S rRNA in the resolved ribosome structure (FIG. 4B). Moreover, examining individual mRNAs bound by HNRNPC (FIG. 4C) or PTBP1 (FIG. 4F) showed that the precise binding site corresponds to the known motif sequence. When we computed this enrichment more globally, we observed that HNRNPC (FIG. 4F) and PTBP1 (FIG. 4F) reads tend to terminate immediately proximal to these well-known binding sequences.

Taken together, the data demonstrates that SPIDR generates highly accurate single nucleotide RBP-binding maps for dozens of RBPs within a single experiment. Moreover, SPIDR can simultaneously map RBPs representing diverse functions and binding modalities, including RBPs that bind within thousands of RNAs (e.g. CPSF6), RBPs that bind only a few very specific RNAs (e.g. SLBP), as well as RBPs that bind primarily within intronic regions within the nucleus (e.g. PTBP1) and RBPs that bind primarily to exonic regions within the cytoplasm (e.g. UPF1).

LARP1 Binds to the 40S Ribosome and mRNAs Encoding Translation-Associated Proteins

FIG. 2A depicts some embodiments of RNA binding patterns of selected RBPs (rows) relative to 100 nt windows across each classical non-coding RNA (columns). Each bin is colored based on the enrichment of read coverage per RBP relative to background.

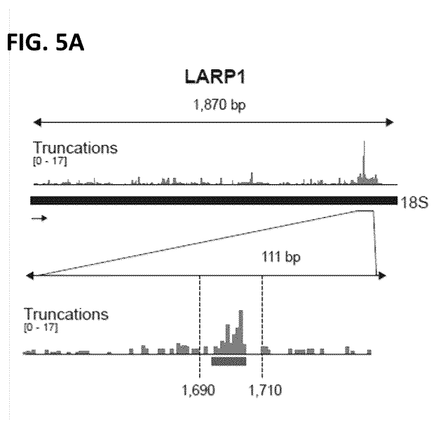

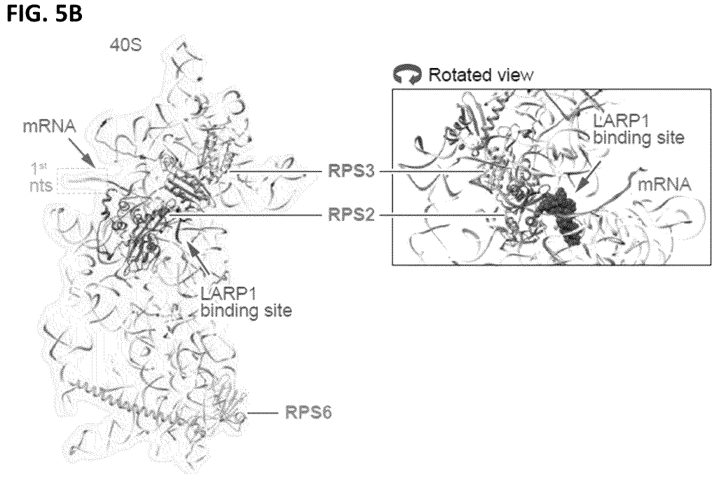

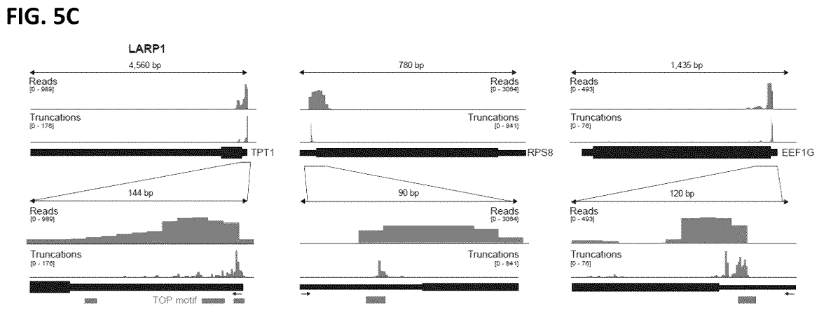

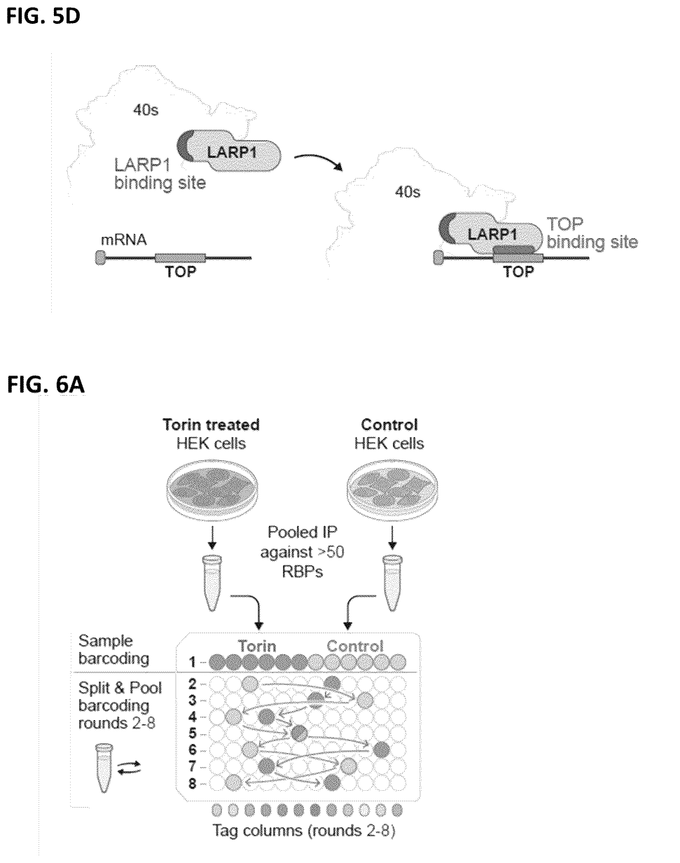

FIG. 5A depicts some embodiments of the frequency of 3′-end truncations of LARP1 reads plotted across the 18S rRNA. Zoom-in shows accumulation near nucleotide position 1700 (indicated by the gray bar). Data shown is from HEK293T cells.

In addition to the three known structural components of the small ribosomal subunit (RPS2, RPS3, and RPS6), LARP1 also showed strong binding to the 18S ribosomal RNA (FIG. 2A, FIG. 5A). LARP1 is an RNA binding protein that has been linked to translational initiation of specific mRNAs. It is known to bind to the 5′ end of specific mRNAs, primarily those encoding critical translation proteins such as ribosomal proteins and initiation and elongation factors, via recognition of a terminal oligopyrimidine (TOP) sequence in the 5′ UTR of these transcripts. The exact role of LARP1 in translation has been debated because it has been reported to both promote and repress translation of mRNAs containing a TOP-motif.

FIG. 5B depicts some embodiments of the structure of the 40S ribosomal subunit bound to the 5′-end of an mRNA molecule. The first nucleotide of the mRNA is indicated as labeled. RPS2 and RPS3 are indicated for orientation and the mRNA is indicated as labeled. green. The LARP1 binding site detected on the 18S rRNA is indicated as labeled.

FIG. 5C depicts some embodiments of examples of LARP1 binding for three different mRNAs containing TOP motifs in their 5′UTRs-TPT1 (left), RPS8 (middle) and EEF1G (right). Both read alignments (“Reads”, top) and 3′end truncations of the cDNA reads (“Truncations”, bottom) are shown. TOP motifs within the 5′-UTRs are depicted as indicated by gray bars on the X-axis.

Although LARP1 is known to bind TOP-motif containing mRNAs, how it might promote translation initiation of these mRNAs is mostly unknown. Because there was a strong binding interaction between LARP1 and the 18S ribosomal RNA, where in the initiating ribosome this interaction occurs was examined. Interestingly, the LARP1 binding site on the 18S ribosomal RNA (1698-1702 nts) is at a distinct location relative to all other 18S binding proteins that were explored and corresponds to a position within the 48S structure that is directly adjacent to the mRNA entry channel (FIG. 5B). More generally, strong binding of LARP1 at the TOP-motif sequence within the 5′ UTR of translation-associated mRNAs (FIG. 5C) was observed.

FIG. 5D depicts some embodiments of a model of LARP1 interactions based on SPIDR data. LARP1 shows preferential binding to both the 18S rRNA (close to the mRNA entry channel of the 40S subunit) and the TOP motifs within the 5′UTR of specific mRNAs. In this way, LARP1 could facilitate recruitment of the 40S subunit to TOP motif-containing mRNAs.

These results suggest that LARP1 may act to promote increased translational initiation of TOP-motif containing mRNAs by directly binding to the 43S pre-initiation complex and recruiting this complex specifically to mRNAs containing a TOP-motif. Because LARP1 is positioned immediately adjacent to the mRNA in this structure, this 43S+LARP1 complex would be ideally positioned to access and bind the TOP motif to facilitate efficient ribosome assembly and translational initiation at these mRNAs. This mechanism of direct ribosome recruitment to TOP-motif containing mRNAs through LARP1 binding to the 43S ribosome and the mRNA would explain why the TOP-motif must be contained within a fixed distance from the 5′ cap to promote translational initiation67 (FIG. 5D).

4EBP1 Binds Specifically to LARP1-Bound mRNAs Upon mTOR Inhibition

Translation of TOP motif-containing mRNAs is selectively repressed upon inhibition of the mTOR kinase, which occurs in conditions of physiological stress. Recent studies have shown that under these conditions, LARP1 binds the 5′-UTR of TOP-containing mRNAs, and it has been postulated that this binding activity is responsible for the specific translational repression of these mRNAs. Yet, the mechanism by which LARP1 binding might repress translation remains unknown.

The canonical model for how mTOR inhibition leads to translational suppression is through the selective phosphorylation of 4EBP1. Specifically, when phosphorylated, 4EBP1 cannot bind to EIF4E, which is the critical initiation factor that binds to the 5′ mRNA cap and recruits the remaining initiation factors through direct binding with EIF4G. When 4EBP1 is not phosphorylated (i.e., in the absence of mTOR), it binds to EIF4E and prevents it from binding to EIF4G and initiating translation. While this differential binding of 4EBP1 to EIF4E upon mTOR modulation is well-established and is central to translational suppression, precisely how it leads to selective modulation of TOP mRNA translation has remained unclear. Specifically, direct competition between 4EBP1 and EIF4G for binding to EIF4E should impact translation of all EIF4E-dependent mRNAs, yet the observed translational downregulation is specific to TOP-containing mRNAs and this specificity is dependent on LARP1 binding.

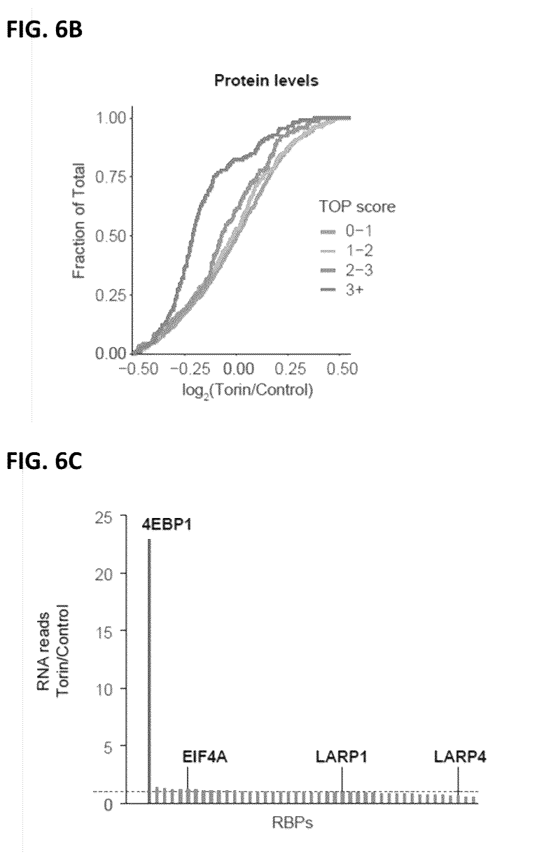

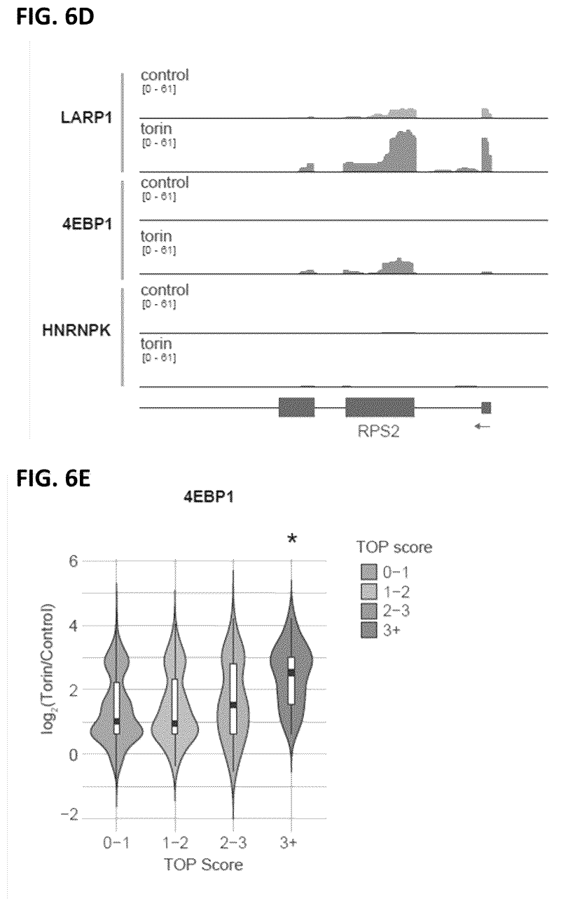

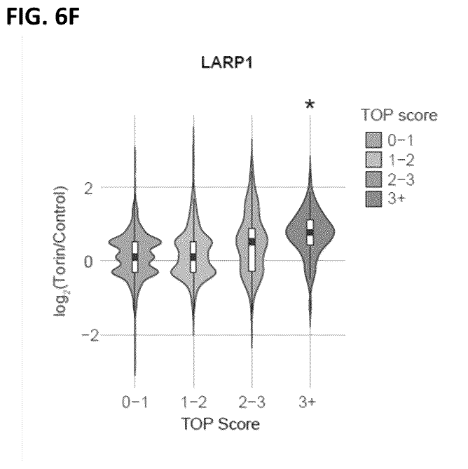

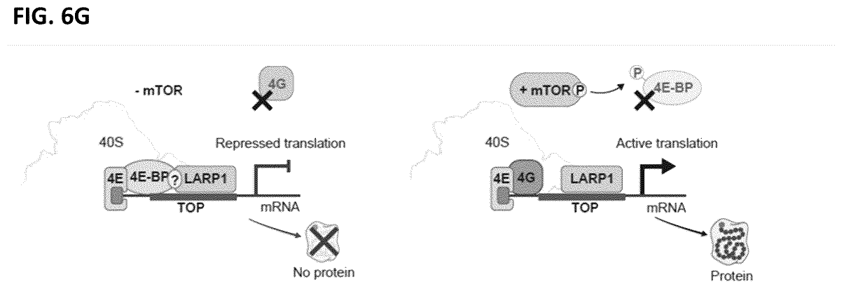

FIG. 6A depicts some embodiments of a schematic of an experimental approach for the mTOR perturbation experiment. HEK293T cells are treated with either 250 nM torin or control (solvent only) for 18 hours. SPIDR is performed on both samples. The multiplexed IP is performed separately, and the samples are mixed after the first round of barcoding.

FIG. 15 is a table depicting some embodiments of an overview of the SPIDR experiment HEK293T cells treated with Torin or Control (solvent only). The protein targets are listed, as well as the vendors and product numbers of the corresponding antibodies.

To explore the mechanism of translational suppression of TOP-containing mRNAs upon mTOR inhibition, HEK293T cells were treated with torin, a drug that inhibits mTOR kinase. SPIDR was adapted to map multiple independent samples within a single split- and -pool barcoding experiment (FIG. 6A, FIG. 15; see Materials and Methods, below, for details) and used this approach to perform SPIDR on >50 distinct RBPs, including LARP1, numerous translational initiation factors, and 4 negative controls in both torin-treated and untreated conditions.

To ensure that mTOR inhibition robustly leads to translational suppression of TOP-containing mRNAs, global protein levels in torin-treated and untreated cells were quantified using quantitative mass spectrometry (see Materials and Methods, below, for details) to determine protein level changes globally. Although the level of most proteins does not change upon torin-treatment, a striking reduction of proteins encoded from TOP motif-containing mRNAs was observed. Indeed, this translational suppression was directly proportional to the strength of the TOP-motif contained within the 5′-UTR of each mRNA (FIG. 6B).

FIG. 6B depicts some embodiments of cumulative Distribution Function (CDF) plots of protein changes in torin versus control treated samples as determined by LC-MS/MS. log 2 ratios (Torin/Control) are shown on the x-axis and fraction of total (from 0 to 1) is shown on the y-axis. Proteins were grouped into four categories based on their TOP motif score as previously published in (Philippe et al., 2020). The analysis was performed on the 2000 most highly expressed genes (based on RNA expression).

FIG. 6C depicts some embodiments of the number of SPIDR reads assigned to each RBP in the torin-treated samples versus control samples. 4EBP1, EIF4A, LARP1 and LARP4 are also indicated. Dashed line corresponds to enrichment of 1.

FIG. 6D depicts some embodiments of raw alignment data for selected RBPs across RPS2, an mRNA with a strong TOP motif. For each protein “control” and “torin” treatment tracks are shown.

FIG. 6E depicts some embodiments of violin plots of the log 2 ratios (torin/control) of significant binding sites for 4EBP1. The RNA targets are grouped based on their TOP motif score as published in (Philippe et al., 2020). Asterisks indicate statistical significance (p-value <0.00001, Mann-Whitney).

Next, changes in RBP binding upon mTOR inhibition was examined. The number of RNA reads observed for each protein upon torin treatment relative to control was measured. While the majority of proteins showed no change in the number of RNA reads, the sole exception was 4EBP1, which showed a dramatic increase (>20-fold) in the overall number of RNA reads produced upon mTOR inhibition (FIG. 6C). Interestingly, this increase corresponded to increased binding specifically at mRNAs containing a TOP-motif (p-value <8×10-10, Mann-Whitney, FIG. 6D and FIG. 6E). Notably, this did not simply reflect an increased level of 4EBP1 binding at the same sites, but instead corresponded to the detection of many statistically significant binding sites only upon mTOR inhibition that were not observed in the presence of mTOR activity (control samples). Consistent with these observations, a previous study observed that 4EBP1 can be in proximity to translationally suppressed mRNAs upon mTOR inhibition.

FIG. 6F depicts some embodiments of violin plots of the log 2 ratios (Torin/Control) of significant binding sites for LARP1. The RNA targets were grouped based on their TOP motif score as published in (Philippe et al., 2020) 60. Asterisks indicate statistical significance (p-value <0.00001, Mann-Whitney).

In contrast to 4EBP1, which showed a dramatic transition in binding activity to mRNA upon mTOR inhibition, no global change in the number of RNA reads purified by LARP1 upon mTOR inhibition was observed (FIG. 6C). Indeed, in both torin-treated and untreated samples strong binding of LARP1 to TOP motif mRNAs as well as to the 18S ribosomal RNA was observed suggesting that this interaction with the 40S ribosome and TOP mRNAs occurs independently of mTOR activity. However, a 1.7-fold increase in levels of binding of LARP1 at TOP mRNAs upon mTOR inhibition (p-value <5.4×10-16, Mann-Whitney, FIG. 6D and FIG. 6F) was observed. This increased enrichment at TOP mRNAs could reflect more LARP1 binding at these specific mRNAs or could reflect the fact that the LARP1 complex might be more stably associated with each mRNA due to translational repression.

FIG. 6G depicts some embodiments of a model of mTOR-dependent repression of mRNA translation. LARP1 binds to the 40S ribosome and to 5′ untranslated region of TOP-containing mRNAs independent of mTOR activity. When mTOR is active (i.e., in the absence of torin; right side), this dual binding modality can recruit the ribosome specifically to TOP-containing mRNAs and promote their translation. When mTOR is inactive (i.e., in the presence of torin), 4EBP1 can bind to TOP-containing mRNAs (possibly through an interaction with LARP1) and to EIF4E. The interaction between 4EBP1 and EIF4E prevents binding between EIF4E and EIF4G, which is required to initiate translation. In this way, LARP1/4EBP1 binding specifically to TOP-containing mRNAs would enable sequence-specific repression of translation.

Together, these results suggest a model that may reconcile the apparently divergent perspectives about the role of LARP1 as both an activator and repressor of translational initiation and explains how selective mTOR-dependent translational repression is achieved (FIG. 6G). Specifically, LARP1 binds to the 40S ribosome and 5′ untranslated region of mRNAs containing a TOP motif regardless of mTOR activity. In the presence of mTOR (FIG. 6G, right side), this dual binding modality can act to promote ribosome recruitment specifically to TOP-containing mRNAs and promote translation of these mRNAs. In the absence of mTOR (FIG. 6G, left side), 4EBP1 can bind to TOP-containing mRNAs, potentially via the LARP1 protein already bound to these mRNAs. Indeed, most of the significant 4EBP1 binding sites are also bound by LARP1 under Torin treatment (60% overlap, odds-ratio of 12-fold, hypergeometric p-value <10-100). By binding selectively to these TOP-containing mRNAs, 4EBP1 can bind to EIF4E and prevent binding between EIF4E and EIF4G, a necessary requirement for initiation of translation. In this way, LARP1/4EBP1 binding to specific mRNAs would enable sequence-specific repression of mRNA translation. This model would explain the apparently divergent roles of LARP1 as both an activator and repressor of translation as it indicates that LARP1 may act as a selective recruitment platform that can either activate or repress translation through the distinct factors that co-bind in the presence or absence of mTOR activity.

### Materials and Methods

### Experimental Conditions

Cell culture K562 cells (ATCC, CCL-243) and HEK293T cells (ATCC, CRL-3216) were purchased from ATCC and cultured under standard conditions. K562 cells were cultured in K562 media consisting of 1× DMEM (Gibco), 1 mM Sodium Pyruvate (Gibco), 2 mM L-Glutamine (Gibco), 1× FBS (Seradigm), 100 U/mL Penicillin-Streptomycin (Life Technologies). HEK293T cells were cultured in HEK293T media consisting of 1× DMEM media (Gibco), 1 mM MEM non-essential amino acids (Gibco), 1 mM Sodium Pyruvate (Gibco), 2 mM L-Glutamine (Gibco), 1× FBS (Seradigm).

### UV-Crosslinking

Crosslinking was performed as previously described23. Briefly, K562 cells were washed once with 1×PBS and diluted to a density of ˜10 million cells/mL in 1×PBS for plating onto culture dishes. HEK293T cells were washed once with 1×PBS and crosslinked directly on culture dishes. RNA-protein interactions were crosslinked on ice using 0.25 J cm-2 (UV 2.5 k) of UV at 254 nm in a Spectrolinker UV Crosslinker. Cells were then scraped from culture dishes, washed once with 1×PBS, pelleted by centrifugation at 330×g for 3 minutes, and flash-frozen in liquid nitrogen for storage at −80° C.

Torin-1 treatment HEK293T cells were treated at a final concentration of 250 nM Torin-1 (Cell Signaling Technology, #14379) in standard HEK293T media for 18 hours prior to UV-crosslinking and harvesting.

### Bead Biotinylation

The bead labeling strategy was adapted from ChIP DIP, a Guttman lab protocol used for multiplexed mapping of hundreds of proteins the DNA (https://guttmanlab.caltech.edu/technologies/). Specifically, 1 mL of Protein G Dynabeads (ThermoFisher, #10003D) were washed once with 1×PBST (1× PBS+0.1% Tween-20) and resuspended in 1 mL PBST. Beads were then incubated with 20 μL of 5 mM EZ-Link Sulfo-NHS-Biotin (Thermo, #21217) on a HulaMixer for 30 minutes at room temperature. Following NHS reaction, beads were placed on a magnet and 500 μL of buffer was removed and replaced with 500 μL of 1M Tris pH 7.4 to quench the reaction for an additional 30 minutes at room temperature. Beads were then washed twice with 1 mL PBST and resuspended in their original storage buffer until use.

Labeling Biotinylated Beads with Oligonucleotide Tags

Unique biotinylated oligonucleotides were first coupled to streptavidin (BioLegend, #280302) in a 96-well PCR plate. In each well, 20 μL of 10 μM oligo was added to 75 μL 1× PBS and 5 μL 1 mg/mL streptavidin. The 96-well plate was then incubated with shaking at 1600 rpm on a ThermoMixer for 30 minutes at room temperature. Each well was then diluted 1:4 in 1×PBS for a final concentration of 227 nM.

For each experiment, the appropriate amount of biotinylated Protein G beads (10 μL beads per capture antibody) was washed once in 1×PBST. Beads were then resuspended in oligo binding buffer (0.5× PBST, 5 mM Tris pH 8.0, 0.5 mM EDTA, 1M NaCl). 200 μL of the bead suspension was aliquoted into individual wells of a 96-well plate, followed by addition of 4 μL of 227 nM streptavidin-coupled oligo to each well. The 96-well plate was then incubated with shaking at 1200 rpm on a ThermoMixer for 30 minutes at room temperature. Beads were then washed twice with M2 buffer (20 mM Tris 7.5, 50 mM NaCl, 0.2% Triton X-100, 0.2% Na-Deoxycholate, 0.2% NP-40), twice with 1×PBST, and resuspended in 200 μL of 1×PBST.

### Binding Antibody to Labeled Protein G Beads

2.5 μg of each capture antibody was added to each well of the 96-well plate containing labeled beads in 1×PBST. The plate was incubated with shaking at 1200 rpm on a ThermoMixer for 30 minutes at room temperature. After incubation, beads were washed twice with 1×PBST+2 mM biotin (Sigma, #B4639-5G), resuspended in 200 μL of 1× PBST+2 mM biotin, and left shaking at 1200 rpm for 10 minutes at room temperature. All wells containing beads were then pooled together and washed twice with 1 mL 1× PBST+2 mM biotin. At this stage, each bead in the bead pool contains a single type of capture antibody with a corresponding unique oligonucleotide tag.

### Pooled Immunoprecipitation

For each experiment, 10 million cells were lysed in 1 mL RIPA buffer (50 mM HEPES pH 7.4, 100 mM NaCl, 1% NP-40, 0.5% Na-Deoxycholate, 0.1% SDS) supplemented with 20 μL Protease Inhibitor Cocktail (Sigma, #P8340-5 mL), 10 μL of Turbo DNase (Invitrogen, #AM2238), 1× Manganese/Calcium mix (2.5 mM MnCl2, 0.5 mM CaCl2), and 5 μL of RiboLock RNase Inhibitor (Thermo Fisher, #EO0382)). Samples were incubated on ice for 10 minutes to allow lysis to proceed. After lysis, cells were sonicated at 3-4 W of power for 3 minutes (pulses 0.7 s on, 3.3 s off) using the Branson sonicator and then incubated at 37° C. for 10 minutes to allow for DNase digestion. DNase reaction was quenched with addition of 0.25 M EDTA/EGTA mix for a final concentration of 10 mM EDTA/EGTA. RNase If (NEB, #M0243L) was then added at a 1:500 dilution and samples were incubated at 37° C. for 10 minutes to allow partial fragmentation of RNA to obtain RNAs of approximately ˜300-400 bp in length. RNase reaction was quenched with addition of 500 μL ice cold RIPA buffer supplemented with 20 μL Protease Inhibitor Cocktail and 5 μL of RiboLock RNase Inhibitor, followed by incubation on ice for 3 minutes. Lysates were then cleared by centrifugation at 15000×g at 4° C. for 2 minutes. The supernatant was transferred to new tubes and diluted in additional RIPA buffer such that the final volume corresponded to 1 mL lysate for every 100 μL of Protein G beads used. Lysate was then combined with the labeled antibody-bead pool and 1 M biotin was added to a final concentration of 10 mM as to quench any disassociated streptavidin-coupled oligos. Beads were left rotating overnight at 4° C. on a HulaMixer. Following immunoprecipitation, beads were washed twice with RIPA buffer, twice with high salt wash buffer (50 mM HEPES pH 7.4, 1 M NaCl, 1% NP-40, 0.5% Na-Deoxycholate, 0.1% SDS), and twice with Tween buffer (50 mM HEPES pH 7.4, 0.1% Tween-20).

### Ligation of the RNA Phosphate Modified (“RPM”) Tag

After immunoprecipitation, 3′ ends of RNA were modified to have 3′ OH groups compatible for ligation using T4 Polynucleotide Kinase (NEB, #M0201L). Beads were incubated at 37° C. for 10 minutes with shaking at 1200 rpm on a ThermoMixer. Following end repair, beads were buffer exchanged by washing twice with high salt wash buffer and twice with Tween buffer. RNA is subsequently ligated with an “RNA Phosphate Modified” (RPM) adaptor (Quinodoz et al 2021) using High ConcentrationT4 RNA Ligase I (NEB, M0437M). Beads were incubated at 24° C. for 1 hour 15 minutes with shaking at 1400 rpm, followed by three washes in Tween buffer. After RPM ligation, RNA was converted to cDNA using SuperScript III (Invitrogen, #18080093) at 42° C. for 20 minutes using the “RPM Bottom” RT primer to facilitate on-bead library construction and a 5′ sticky end to ligate tags during split-and-pool barcoding. Excess primer is digested with Exonuclease I (NEB, #M0293L) at 37° C. for 15 minutes.

### Split-and-Pool Barcoding to Identify RNA-Protein Interactions

Split-and-pool barcoding was performed. Specifically, beads were split-and-pool ligated over ≥6 rounds with a set of “Odd,” “Even,” and “Terminal” tags. The number of barcoding rounds performed for each SPIDR experiment was determined based on the complexity of the given bead pool. All split-and-pool ligation steps were performed for 5 minutes at room temperature and supplemented with 2 mM biotin and 1:40 RiboLock RNase Inhibitor to prevent RNA degradation. We ensured that virtually all barcode clusters (>95%) represented molecules belonging to unique, individual beads.

Compared to previously published approaches, the number of barcodes per round was reduced, but the rounds of split and pool barcoding was increased. Therefore, the barcoding procedure was significantly simplified in contrast to previous versions. For example, for the K562 cells pooled experiment, 6 rounds of 24 barcodes were used for combinatorial barcoding (with a scheme of Odd, Even, Odd, Even, Odd, Terminal tag). For the HEK293T cells mTOR inhibition experiment, 6 rounds of 36 barcodes were used for combinatorial barcoding to achieve sufficient barcode complexity. Of the 36 barcodes used in round one of the ligations, 18 were used to label the control condition and the remaining 18 were used to label the torin treated condition. The samples were then pooled together for the remaining 5 rounds of ligation.

### Library Preparation

After split-and-pool barcoding, beads were aliquoted into 5% aliquots for library preparation and sequencing. RNA in each aliquot was degraded by incubating with RNase H (NEB, #M0297L) and RNase cocktail (Invitrogen, #AM2286) at 37° C. for 20 minutes. 3′ ends of the resulting cDNA were ligated to attach dsDNA oligos containing library amplification sequences using a “splint” ligation as previously described (Quinodoz et al 2021) 31. The “splint” ligation reaction was performed with 1× Instant Sticky End Master Mix (NEB #M0370) at 24° C. for 1 hour with shaking at 1400 rpm on a ThermoMixer. Barcoded cDNA and biotinylated oligo tags were then eluted from beads by boiling in NLS elution buffer (20 mM Tris-HCl pH 7.5, 10 mM EDTA, 2% N-lauroylsarcosine, 2.5 mM TCEP) for 6 minutes at 91° C., with shaking at 1350 rpm.

Biotinylated oligo tags were first captured by diluting the eluant in 1× oligo binding buffer (0.5× PBST, 5 mM Tris pH 8.0, 0.5 mM EDTA, 1M NaCl) and subsequently binding to MyOne Streptavidin C1 Dynabeads (Invitrogen, #65001) at room temperature for 30 minutes. Beads were placed on a magnet and the supernatant, containing cDNA, was moved to a separate tube. Biotinylated oligo tags were amplified on-bead using 2× Q5 Hot-Start Mastermix (NEB #M0494) with primers that add the indexed full Illumina adaptor sequences.

To isolate barcoded cDNA, the supernatant was first incubated with a biotinylated antisense ssDNA (“anti-RPM”) probe that hybridizes to the junction between the reverse transcription primer and splint sequences to reduce empty insertion products. This mixture was then bound to MyOne Streptavidin C1 Dynabeads at room temperature for 30 minutes. Beads were placed on a magnet and the supernatant, containing the remaining cDNA products, was cleaned up on Silane beads (Invitrogen, #37002D) as previously described83. Finally, cDNA was amplified using 2× Q5 Hot-Start Mastermix (NEB #M0494) with primers that add the indexed full Illumina adaptor sequences.

After amplification, libraries were cleaned up using 1× SPRI (AMPure XP), size-selected on a 2% agarose gel, and cut at either ˜300 nt (barcoded oligo tag) or between 300-1000 nt (barcoded cDNA). Libraries were subsequently purified with Zymoclean Gel DNA Recovery Kit (Zymo Rescarch, #4007).

Paired-end sequencing was performed on either an Illumina NovaSeq 6000 (S4 flowcell), NextSeq 550, or NextSeq 2000 with read lengths ≥100×200 nucleotides. For the K562 data, 37 SPIDR aliquots were generated and sequenced from two technical replicate experiments. The two experiments were generated using the same batch of UV-crosslinked lysate processed on the same day. For the HEK293T data, 9 SPIDR aliquots were generated from a single technical replicate. Each SPIDR library corresponds to a distinct aliquot that was separately amplified with different indexed primers, providing an additional round of barcoding as previously described31. Minimum required sequencing depth for each experiment was determined by the estimated number of beads and unique molecules in each aliquot. For oligo tag libraries, each library was sequenced to a depth of observing ˜5 unique oligo tags per bead on average. For cDNA libraries, each library was sequenced with at least 2× coverage of the total estimated library complexity.

### Analysis and Processing Pipeline

### Read Processing and Alignment

Paired-end RNA sequencing reads were trimmed to remove adaptor sequences using Trim Galore! v0.6.2 and assessed with FastQC v0.11.8. Subsequently, the RPM (ATCAGCACTTA) sequence was trimmed using Cutadapt v3.4 from both 5′ and 3′ read ends. The barcodes of trimmed reads were identified with Barcode ID v1.2.0 (https://github.com/GuttmanLab/sprite2.0-pipeline) and the ligation efficiency was assessed. Reads with or without an RPM sequence were split into two separate files to process RNA and oligo tag reads individually downstream, respectively.

RNA read pairs were then aligned to a combined genome reference containing the sequences of repetitive and structural RNAs (ribosomal RNAs, snRNAs, snoRNAs, 45S pre-rRNAs, tRNAs) using Bowtie2. The remaining reads were then aligned to the human (hg38) genome using STAR aligner. Only reads that mapped uniquely to the genome were kept for further analysis.

Barcode matching and filtering Mapped RNA and oligo tag reads were merged, and a cluster file was generated for all downstream analysis as previously described. MultiQC v1.6 was used to aggregate all reports. To unambiguously exclude ligation events that could not have occurred sequentially, we utilized unique sets of barcodes for each round of split-and-pool. All clusters containing barcode strings that were out-of-order or contained identical repeats of barcodes were filtered from the merged cluster file. To determine the amount of unique oligo tags present in each cluster, sequences sharing the same Unique Molecular Identifier (UMI) were removed and the remaining occurrences were counted. To remove PCR duplication events within the RNA library, sequences sharing identical start and stop genomic positions were removed.

### Splitting Alignment Files by Protein Identity

Barcode strings from filtered cluster files were then used to assign protein identities to the alignment file containing all mapped RNA reads. Because each cluster represents an individual bead, the frequency of oligo tags (each representing unique protein type) was used to determine protein assignments. Specifically, for each cluster we required ≥3 observed oligo tags and that the most common protein type represented ≥80% of all observed tags. RNA reads were then split into separate alignment files by barcode strings corresponding to protein type.

### Background Correction and Peak Calling

In order to determine what portion of the observed signal is specific to a particular capture antibody, rather than common pileups regardless of the protein captured, coverage was normalized for each protein relative to the coverage detected for all other proteins. Specifically, for a protein of interest, we computed the number of reads that were mapped to that protein. All reads not assigned to that protein were randomly down sampled such that it had a comparable number of reads as the protein of interest. To measure the expected variance in the control sample, we repeated this down sampling procedure at least 100 independent times. We then computed read counts per window across the transcriptome (either 10 nts or 100 nts) for the protein of interest and each of the randomized control samples. We computed a normalized enrichment as the number of observed reads within the window (observed) divided by the average of the read counts overt that window across the >=100 permutations (expected). To assess the significance of this enrichment score, we measured how often the observed score was seen in the >=100 permutations. A p-value was assigned as the number of random scores greater than or equal to the observed scores divided by the number of random permutations used (we included the actual observed score in the numerator and denominator). All windows that had at least 10 observed reads and a p-value less than 0.05 were considered significantly enriched.

### Peak Annotation

Enriched windows were first filtered to only include regions resulting from reads that could be uniquely mapped in the second STAR alignment, and then poor alignments to rRNA regions (chr21: 88206400-8449330) were removed. These filtered peaks were then annotated based on overlap with GENCODE v41 transcripts. In the case of overlapping annotations, the final assigned annotation was chosen based on the following priority list: miRNA, CDS, 5′UTR, 3′UTR, proximal intron (within 500 nt of the splice site region), distal intron (further than 500 nt of the splice site region), non-coding exon, and finally non-coding intron. Windows for which the primary gene annotation was a miRNA host gene were marked as miRNA proximal.

### SPIDR Comparison to ENCODE

### ENCODE Datasets

43 of the proteins included in SPIDR also had a matched K562 ENCODE eCLIP experiment with paired-end sequencing data. The raw FASTQ files for these datasets were downloaded from the ENCODE website (https://www.encodeproject.org/) and aligned to the genome using the same parameters as in the SPIDR dataset.

For comparison of matched SPIDR and ENCODE datasets, the larger of the pair of alignment files was down sampled to the depth of the smaller alignment file. Windows of enrichment in ENCODE datasets were then determined using the same background correction strategy and thresholding as in SPIDR (minimum read count of 10, p-value <0.05). As was done in the SPIDR data, all ENCODE datasets were used as negative controls for one for one another when determining background correction factors and calling windows of enrichment.

### Motif Enrichment Analysis

Filtered SPIDR peaks were used to subset the corresponding SPIDR alignment files, such that only reads that fell within enriched windows were kept. These reads were then used as input for de novo motif analysis by HOMER (http://homer.ucsd.edu/homer/). Motifs with a reported p-value <10-40 were considered significant.

### Comparison of Bound RNA Features

Enriched windows for both SPIDR and ENCODE, as determined using the SPIDR workflow of background correction and thresholding, were annotated based on overlap with GENCODE v41 transcripts. Peaks annotated as intergenic were removed, and then both the SPIDR and ENCODE datasets were filtered to include only proteins that had greater than 100 peaks.

The likelihood of seeing a similarity between SPIDR and ENCODE in the region annotations is visualized by comparing the observed values to randomly shuffled values. The inputs for this method are two matrices, one for SPIDR and one for ENCODE, with the percentage of annotations observed for a given region type for a given RBP. Shuffling is performed by randomly switching percentages across RBPs, keeping the relative values between regions constant. This can be thought of as randomly shuffling the columns of one of the input matrices. A distance is calculated by flattening the two input matrices into vectors, taking the difference between the two vectors, and calculating an L2-norm on that difference. In Supplemental FIG. 7 the histogram of L2-norms shows the distribution we would expect if RBPs had no effect on the L2-norm between SPIDR and ENCODE. The dashed vertical line represents the L2-norm when the input matrices were flattened but not shuffled.

The basic algorithm is as follows:
Calculate the true L2-norm between SPIDR and ENCODE.
Keeping SPIDR constant, randomly switch probabilities between RBPs while keeping the percentages within an RBP the same for ENCODE.
Repeat step 2, shuffling SPIDR and keeping ENCODE constant.
Repeat steps 2 and 3 for 1000 samples.

### Single Nucleotide Resolution Analysis

We computed the frequency of reads ending at the 3′ end of the cDNA. We computed enrichment for each of these counts by randomly down sampling all reads not assigned to the specific protein and computing the same 3′ end coverage. Enrichments and p-values were computed as described above and as previously reported in (Banerjee et al. 2020) 84.

mTOR Analysis

Background corrected bedgraphs were generated from control and +Torin conditions for each RBP in each condition. These bedgraph values were then mapped on to Refseq genes using the bedtools map command (arguments: -c 4-0 absmax). Where multiple isoforms were present for the same gene, the isoform with the highest map count was used. To normalize for possible detection bias due to fewer antibody beads in one condition versus the other we adjusted the map value by the ratio of antibody beads as determined by number of bead clusters corresponding to each antibody in each respective condition. Number of antibody (bead clusters) were defined and calculated using the same values used to generate the split bam files for each protein (options: minimum number of oligos=3, fraction unique=0.8, max number of RNAs in clusters=100). The ratio of cluster-corrected values for each gene across the two conditions was then compared per gene and separated based on TOP score. Published TOP scores60 were used to generate categories for violin plots.

For the protein changes CDF plots, we first selected for the 2000 highest expressed genes based on previous RNA-seq data84. Input TPM values for HEK293 cells were taken from input CLAP (sub_input.merged.bam) data from HEK293T cell in (Banerjee et al. 2020). The input samples were down sampled to 20M reads prior to TPM calculation. Feature counts was used to calculate read overlaps with hg38 protein coding refseq genes and further converted to TPM values. The top 2000 expressed genes (based on HEK293 input TPM) were used to plot the average protein log 2 fold changes (Torin versus control) vs TOP score. Published TOP scores60 were used to plot CDF values.

### Mass Spectrometry

### Multiplexed Immunopurification (IP) for Mass Spectrometry

10 million K562 cells were lysed in 4 mL of RIPA on ice for 10 minutes. The lysate was clarified by centrifugation at 15000 g for 2 minutes, and then split in half for either the pooled IP with 39 antibodies or the negative control IP with an anti-V5 antibody. Each half of the lysate was combined with 10 μg total antibody (0.25 μg per each antibody for the pooled IP) and 100 μL of Protein G beads and left rotating at 4C overnight. The beads were then washed twice with RIPA, twice with High Salt Wash Buffer, twice with Clap-Tween, and finally three times with Mass Spec IP Wash Buffer (150 mM NaCl, 50 mM Tris-HCl pH 7.5, 5% Glycerol). Each sample was then reduced, alkylated, Trypsin digested, and desalted as described in (Parnas et al, 2015) 85. Peptides were reconstituted in 12 μL 3% acetonitrile/0.1% formic acid.

mTOR Proteomics

5 million cells each of control and 250 nM Torin-1 treated HEK cells were lysed in 250 μL Mass Spec Lysis Buffer (8M urea, 75 mM NaCl, 50 mM Tris pH 8.0, 1 mM EDTA) for 30 min at room temperature. Samples were then clarified by centrifugation at 23000 g for 5 minutes, and the protein content in the supernatant was measured by BCA assay (ThermoFisher, #PI23227). 40 μg of protein for each sample was reduced with 5 mM final dithiothreitol (DTT) for 45 minutes at room temperature and subsequently alkylated with 10 mM final iodoacetamide (IAA) for 45 minutes in the dark at room temperature. 50 mM Tris (pH 8.0) was then added to each sample such that the final concentration of urea was less than 2M. Samples were digested overnight with 0.4 μg Trypsin (Promega, #V5113) for a 1:100 enzyme to protein ratio. Peptides were desalted on C18 StageTips according to (Rappsilber et al., 2007) 86.

LC-MS/MS LC-MS/MS analysis was performed on a Q-Exactive HF. 5 μL of total peptides were analyzed on a Waters M-Class UPLC using a C18 25 cm Thermo EASY-Spray column (2 um, 100A, 75 um×25 cm) or IonOpticks Aurora ultimate column (1.7 um, 75 um×25 cm) coupled to a benchtop ThermoFisher Scientific Orbitrap Q Exactive HF mass spectrometer. Peptides were separated at a flow rate of 400 nL/min with a linear 95 min gradient from 5% to 22% solvent B (100% acetonitrile, 0.1% formic acid), followed by a linear 30 min gradient from 22 to 90% solvent B. Each sample was run for 160 min, including sample loading and column equilibration times. Data was acquired using Xcalibur 4.1 software.

The IP samples were measured in a Data Dependent Acquisition (DDA) mode. MS1 Spectra were measured with a resolution of 120,000, an AGC target of 3e6 and a mass range from 300 to 1800 m/z. Up to 12 MS2 spectra per duty cycle were triggered at a resolution of 15,000, an AGC target of 1e5, an isolation window of 1.6 m/z and a normalized collision energy of 28.

The Torin treated and control total lysate samples were measured in a Data Independent Acquisition (DIA) mode. MS1 Spectra were measured with a resolution of 120,000, an AGC target of 5e6 and a mass range from 350 to 1650 m/z. 47 isolation windows of 28 m/z were measured at a resolution of 30,000, an AGC target of 3e6, normalized collision energies of 22.5, 25, 27.5, and a fixed first mass of 200 m/z.

Database searching of the proteomics raw files Proteomics raw files were analyzed using the directDIA method on SpectroNaut v16.0 for DIA runs or SpectroMine (3.2.220222.52329) for DDA runs (Biognosys) using a human UniProt database (Homo sapiens, UP000005640), under BSG factory settings, with automatic cross-run median normalization and imputation. Protein group data were exported for subsequent analysis.

What is claimed is:

1. A method of detecting an association between a RNA binding protein and a RNA, the method comprising:
a. providing an antibody-bead conjugate pool comprising a plurality of antibody-bead conjugate populations, wherein
each antibody-bead conjugate population comprises a plurality of antibody-bead conjugates, wherein
each antibody-bead conjugate in an antibody-bead conjugate population comprises an antibody specific for the same RNA binding protein and an RBP-identifying oligonucleotide, wherein
each antibody-bead conjugate population in the antibody-conjugate pool comprises antibodies specific for a different RNA binding protein;
b. providing a composition comprising a plurality of crosslinked RNA: RBP complexes,
c. immunopurifying the cross-linked RNA using the antibody-bead conjugate pool,
d. performing one or more rounds of split-and-pool barcoding, wherein during each of the one or more rounds of split-and-pool barcoding the same barcode oligonucleotide is added to both the RBP identifying oligonucleotide and the immunopurified RNA on each antibody-bead conjugate;
e. sequencing the barcoded molecules, and
f. assigning the one or more RNA molecules to their corresponding RNA binding protein.

2. The method of claim 1, wherein the antibody-bead conjugate pool comprises a biotinylated protein G bead bound to a streptavidin-biotin-tag complex.

3. The method of claim 1, wherein generating an antibody-bead conjugate pool comprises:
a. incubating one or more populations of biotinylated protein G beads with a streptavidin-biotin oligo complex, wherein
each population of beads is labeled with an antibody identifying oligonucleotide;
b. incubating each of the one or more populations of beads with an antibody, wherein
each population of beads is incubated with a different antibody; and
c. combining each of the one or more populations of beads to generate an antibody-bead conjugate pool.

4. The method of claim 1, wherein the bead comprises a protein A, protein G, or protein A/G bead.

5. The method of claim 1, wherein the composition comprises a cell.

6. The method of claim 5, further comprising lysing the cell.

7. The method of claim 1, wherein the composition comprises a cell lysate.

8. The method of claim 1, wherein providing a composition comprising crosslinked RNA and RNA binding proteins comprises applying a crosslinking agent to a composition comprising a plurality of RNA molecules and a plurality of RNA binding proteins.

9. The method of claim 1, wherein assigning the one or RNA to their corresponding RNA binding protein comprises matching the antibody-bead conjugate and RNA based on their shared barcode oligonucleotides.

10. The method of claim 1, wherein immunopurifying the cross-linked RNA using the antibody-bead conjugate pool enriches the RNA binding protein by at least about 2-fold relative to a negative control.

11. The method of claim 1, wherein up to 10 rounds of split-and-pool barcoding are performed.

12. The method of claim 1, wherein up to unique 20 barcodes are used in each round of split-and-pool barcoding.

13. The method of claim 1, wherein at least 10 different RBP: RNA interactions are identified.

14. An antibody-bead conjugate population, the antibody-bead conjugate population comprising a plurality of antibody-bead conjugates, wherein
each bead in the antibody-bead conjugate population is conjugated to an antibody or binding fragment thereof specific for a RNA binding protein, wherein
each antibody or binding fragment thereof in the antibody-bead conjugate population is specific for the same RNA binding protein;
each bead in the antibody-bead conjugate population is labeled with the same RNA binding protein-identifying oligonucleotide, wherein

15. The method of claim 14, wherein the bead comprises a protein A, protein G, or protein A/G bead.

16. The method of claim 14, wherein the bead is a magnetic bead.

17. An antibody-bead conjugate pool, the antibody-bead conjugate pool comprising a plurality of antibody-bead conjugate populations, wherein
each antibody-bead conjugate population comprises a plurality of antibody-bead conjugates comprising an antibody or binding fragment thereof specific for a single RNA binding protein and an RBP-identifying oligonucleotide, and wherein
each antibody-bead conjugate population in the antibody-conjugate pool is specific for a different RNA binding protein.

18. A kit comprising:
an antibody-bead conjugate pool, wherein
the antibody-bead conjugate pool comprises a plurality of antibody-bead conjugate populations, wherein
each antibody-bead conjugate population in the antibody-bead conjugate pool comprises a different antibody identifying oligonucleotide, wherein
the antibody identifying oligonucleotide is attached to the bead of each antibody-bead conjugate, and
each antibody-bead conjugate population in the antibody-bead conjugate pool is specific for a different RNA binding protein.

19. The kit of claim 18, wherein the antibody-bead conjugate pool comprises a biotinylated protein G bead bound to a streptavidin-biotin-tag complex.

20. The kit of claim 18, further comprising one or more barcode oligonucleotides.

21. The kit of claim 18, wherein the kit comprises up to 100 unique barcode oligonucleotides.

22. The kit of claim 18, further comprising a cross-linking agent.
